SEQUENCE LISTING (26 sequences)

SEQ ID NO: 1 - DNA, 27 bp, synthetic construct
  source 1..27: mol_type = other DNA
tgacggggtcacccacactgtgcccat

SEQ ID NO: 2 - DNA, 30 bp, synthetic construct
  source 1..30: mol_type = other DNA
ctagaagcatttgcggtggacgatggaggg

SEQ ID NO: 3 - DNA, 22 bp, synthetic construct
  source 1..22: mol_type = other DNA
acctgtaacccaaggagatgtg

SEQ ID NO: 4 - DNA, 20 bp, synthetic construct
  source 1..20: mol_type = other DNA
cacaattggcaggaaagtgg

SEQ ID NO: 5 - DNA, 18 bp, synthetic construct
  source 1..18: mol_type = other DNA
aagtgggttagtcggttg

SEQ ID NO: 6 - DNA, 18 bp, synthetic construct
  source 1..18: mol_type = other DNA
ggcagttagctaggtttg

SEQ ID NO: 7 - DNA, 20 bp, synthetic construct
  source 1..20: mol_type = other DNA
tcgctggtctacctgctctt

SEQ ID NO: 8 - DNA, 17 bp, synthetic construct
  source 1..17: mol_type = other DNA
ggcctgtgtgaccttgg

SEQ ID NO: 9 - DNA, 20 bp, synthetic construct
  source 1..20: mol_type = other DNA
cccggagtttgaaaatgaag

SEQ ID NO: 10 - DNA, 23 bp, synthetic construct
  source 1..23: mol_type = other DNA
cagtgcctcttctgaatcaattt

SEQ ID NO: 11 - DNA, 18 bp, synthetic construct
  source 1..18: mol_type = other DNA
agagtcacgctggcaacc

SEQ ID NO: 12 - DNA, 21 bp, synthetic construct
  source 1..21: mol_type = other DNA
ggcagagactctccatcacac

SEQ ID NO: 13 - DNA, 20 bp, synthetic construct
  source 1..20: mol_type = other DNA
agctggatgaccagagtgct

SEQ ID NO: 14 - DNA, 20 bp, synthetic construct
  source 1..20: mol_type = other DNA
tgaaattcatggctgtggaa

SEQ ID NO: 15 - DNA, 20 bp, synthetic construct
  source 1..20: mol_type = other DNA
atgaaggaaaagccaagcag

SEQ ID NO: 16 - DNA, 20 bp, synthetic construct
  source 1..20: mol_type = other DNA
ccaccaaatgtgaagacgtg

SEQ ID NO: 17 - DNA, 21 bp, synthetic construct
  source 1..21: mol_type = other DNA
acatgttcagctttgtggacc

SEQ ID NO: 18 - DNA, 21 bp, synthetic construct
  source 1..21: mol_type = other DNA
tgattggtgggatgtcttcgt

SEQ ID NO: 19 - DNA, 20 bp, synthetic construct
  source 1..20: mol_type = other DNA
atggattcgtggtggaagtg

SEQ ID NO: 20 - DNA, 21 bp, synthetic construct
  source 1..21: mol_type = other DNA
gtggagttcagatgatcagcg

SEQ ID NO: 21 - DNA, 20 bp, synthetic construct
  source 1..20: mol_type = other DNA
gactgtgacgagttggctga

SEQ ID NO: 22 - DNA, 20 bp, synthetic construct
  source 1..20: mol_type = other DNA
ctggagaggagcagaactgg

SEQ ID NO: 23 - DNA, 21 bp, synthetic construct
  source 1..21: mol_type = other DNA
aagtgcggtgcaaactttctt

SEQ ID NO: 24 - DNA, 19 bp, synthetic construct
  source 1..19: mol_type = other DNA
tctcggtggctggtagtga

SEQ ID NO: 25 - DNA, 18 bp, synthetic construct
  source 1..18: mol_type = other DNA
cgggtcctggcatcttgt

SEQ ID NO: 26 - DNA, 21 bp, synthetic construct
  source 1..21: mol_type = other DNA
cagtcttggcagtgcagatga

(19) United States
(12) Patent Application Publication
(10) Pub. No.: US 2026/0297554 A1
(43) Pub. Date: Oct. 1, 2026

(54) METHOD OF DIFFERENTIATING STEM CELLS

(71) Applicant: ROYAL MELBOURNE INSTITUTE OF TECHNOLOGY, Melbourne (AU)

(72) Inventors: Leslie YEO, Melbourne (AU); Lizebona August AMBATTU, Melbourne (AU)

(21) Appl. No.: 19/126,778

(22) PCT Filed: Jan. 11, 2023
(86) PCT No.: PCT/AU2023/050012

§ 371 Date: May 2, 2025

(30) Foreign Application Priority Data
Nov. 4, 2023 (AU) 2022903305

(51) Int. Cl. C12N 13/00; A61K 35/32; C12N 5/077

(52) U.S. Cl. CPC C12N 13/00; A61K 35/32; C12N 5/0654; C12N 2506/1369; C12N 2506/1384; C12N 2521/10

(57) ABSTRACT

The present disclosure relates to methods for differentiating mesenchymal stem cells (MSCs) towards an osteogenic lineage using daily short exposure to acoustic wave energy at a frequency greater than 1 MHz. Methods disclosed herein can be used to obtain a population of osteogenic-committed cells. Osteogenic-committed cells can be used in various cell therapy applications, for example, treatment of bone diseases.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

The present application claims priority from AU 2022903305 filed 4 Nov. 2022, the entire contents of which are incorporated herein by reference.

### FIELD OF THE DISCLOSURE

Embodiments generally relate to methods for differentiating stem cells towards an osteogenic lineage.

### BACKGROUND

Osteoporosis is a chronic skeletal disorder characterised by decreasing bone mass and mineral density. Osteoporosis is more common in elderly people and is responsible for most bone fractures suffered in this population. Osteoporotic hip fractures in particular are associated with high morbidity and mortality. However, there is currently no pharmacological cure for osteoporosis.

Stem cell therapies are considered a promising strategy for treating various types of diseases, particularly degenerative diseases such as osteoporosis. However, methods of manufacturing cells suitable for therapy (i.e. differentiation towards an osteogenic lineage) are incredibly time and cost intensive, and are therefore difficult to upscale.

Accordingly, there remains a need in the art for improved methods of differentiating stem cells towards an osteogenic lineage.

### SUMMARY OF THE DISCLOSURE

The present inventors have shown herein that exposing mesenchymal stem cells to intermittent acoustic wave energy promotes differentiation of the stem cells towards an osteogenic lineage.

Accordingly, in an example, the disclosure provides a method of differentiating a population of mesenchymal stem cells towards an osteogenic lineage, the method comprising exposing the population of stem cells to intermittent short duration acoustic wave energy.

In an example, the population of mesenchymal stem cells are differentiated into osteoblasts.

In an example, the acoustic wave energy is provided at a frequency greater than 1 MHz. In an example, the acoustic wave energy is provided at a frequency of at least about 2 MHz. In an example, the acoustic wave energy is provided at a frequency greater than 3 MHz. In an example, the acoustic wave energy is provided at a frequency between about 1 MHz and about 1 GHz. In an example, the acoustic wave energy is provided at a frequency between greater than 1 MHz and about 1 GHz. In an example, the acoustic wave energy is provided at a frequency between greater than 3 MHz and about 1 GHz. In an example, the acoustic wave energy is provided at a frequency between about 3 MHz and about 50 MHz. In an example, the acoustic wave energy is provided at a frequency between about 4 MHz and about 50 MHz. In an example, the acoustic wave energy is provided at a frequency between about 5 MHz and about 35 MHz. In an example, the acoustic wave energy is provided at a frequency between about 25 MHz and about 35 MHz. In an example, the acoustic wave energy is provided at a frequency between about 5 MHz and about 15 MHz. In an example, the acoustic wave energy is provided at a frequency between about 8 MHz and about 12 MHz. In an example, the acoustic wave energy is provided at a frequency of about 10 MHz.

In an example, the acoustic wave energy is provided in the form of surface reflected bulk waves (SRBW). In an example, when the acoustic wave energy is provided in the form of SRBW the frequency between about 5 MHz and about 15 MHz.

In an example, the acoustic wave energy is provided in the form of surface acoustic waves (SAW). In an example, when the acoustic wave energy is provided in the form of SAW the frequency between about 25 MHz and about 35 MHz.

In an example, the mesenchymal stem cells are bone marrow derived mesenchymal stem cells, adipose derived mesenchymal stem cells, or umbilical cord derived mesenchymal stem cells.

The methods disclosed herein involve exposing stem cells to short duration intermittent exposure, as opposed to continuous exposure over a long duration of time. In an example, the intermittent exposure comprises exposing the population of cells to a one or more periods of acoustic wave energy followed by incubation in the absence of acoustic stimulation for one or more consecutive days. In an example, the short duration comprises a period of between about 3 minutes and about 20 minutes. In an example, the short duration comprises a period of between about 5 minutes and about 15 minutes. In an example, the short duration comprises a period of between about 8 minutes and about 12 minutes. In an example, the cells are exposed to acoustic wave energy for a period of about 10 minutes.

In an example, the cells are exposed to acoustic wave energy for a short duration period once, twice, 3 times, 4 times, 5 times, 6 times, 7 times, 8 times, 9 times, or 10 times per day. In an example, the cells are exposed to acoustic wave energy for a short duration period once per day.

Surprisingly, the method of the invention confers commitment of mesenchymal stem cells towards an osteogenic lineage after a relatively short period. Thus, in an example, the cells are exposed to acoustic wave energy for a short duration period for at least 3 days, for at least 4 days, or for at least 5 days. In an example, the cells are exposed to acoustic wave energy for at least 5 days.

The present inventors have also surprisingly found that a relatively short period of exposure to intermittent short duration acoustic wave energy as defined herein is required to maintain commitment of the mesenchymal stem cells towards an osteogenic lineage. Thus, in an example, the cells are exposed to acoustic wave energy for between 2 days and 10 days. In an example, the cells are exposed to acoustic wave energy for between 3 days and 6 days. In an example, the cells are exposed to acoustic wave energy for 5 days.

In an example, the cells are maintained in culture for a period of between 1 day and 30 days following acoustic wave stimulation. In an example, the cells are maintained in culture for a period of between 5 days and 25 days following acoustic wave stimulation. In an example, the cells are maintained in culture for a period of between 10 days and 20 days following acoustic wave stimulation. In an example, the cells are maintained in culture for a period of 14 days following acoustic wave stimulation. In an example, the cells are maintained in culture for a period of 15 days following acoustic wave stimulation. In an example, the cells are maintained in culture for a period of 16 days following acoustic wave stimulation. In an example, the cells are harvested on the fifth consecutive day of exposure to acoustic wave energy.

In an example, the cells express at least one osteogenic marker after at least 3 consecutive days of exposure to acoustic wave energy. In an example, the cells express at least one osteogenic marker after between 3 consecutive days and 10 consecutive days of exposure to acoustic wave energy. In an example, the cells express at least one osteogenic marker after between 3 consecutive days and 6 consecutive days of exposure to acoustic wave energy. In an example, the cells express at least one osteogenic marker after 5 consecutive days of exposure to acoustic wave energy.

In an example, the osteogenic marker is one or more or all of those selected from group comprising, but not limited to, RUNX2, COL1A1, and OCN.

In an example, the acoustic wave energy is provided to the population of cells via an apparatus, the apparatus comprising: (i) an acoustic wave generator configured to generate acoustic energy at a selected power and frequency; and (ii) a receptacle for accommodating a population of cells in a culture medium, the receptacle being configured to receive acoustic energy generated by the acoustic wave generator.

In an example, at least part of the acoustic wave generator is arranged in direct contact with the culture medium.

In an example, the acoustic wave generator is separated from the culture medium.

In an example, the receptacle defines a reservoir configured to accommodate the population of cells in a culture medium.

In an example, the acoustic wave generator comprises a piezoelectric substrate defining a working surface and an interdigitated transducer located on and in contact with the working surface of the piezoelectric substrate.

In an example, the receptacle is coupled to the acoustic wave generator with a coupling material.

In an example, the acoustic wave energy is propagated as a surface acoustic wave (SAW) along the working surface.

In an example, the acoustic wave energy is propagated as a surface reflected bulk wave (SRBW) within the piezoelectric substrate and internally reflected between the working surface and an adjacent surface of the piezoelectric substrate.

In an example the frequency of the applied acoustic energy is in the range of about 1 MHz to about 1 GHz. In an example, the frequency of the applied acoustic energy is in the range of about 5 MHz to about 40 MHz. In an example, the frequency of the applied acoustic energy is in the range of about 5 MHz to about 35 MHz. In an example, the frequency of the applied acoustic energy is in the range of about 5 MHz to about 20 MHz. In an example, the frequency of the applied acoustic energy is in the range of about 5 MHz to about 15 MHz. In an example, the frequency of the applied acoustic energy is in the range of about 25 MHz to about 35 MHz. In an example, the frequency of the applied acoustic energy is in the range of about 8 MHz to about 12 MHz. In an example, the frequency of the applied acoustic energy is about 10 MHz.

In an example, the input power for the acoustic wave generator is in the range of about 1 W to about 10 W. In an example, the input power for the acoustic wave generator is in the range of about 1 W to about 5 W. In an example, the input power for the acoustic wave generator is in the range of about 1.5 W to about 3 W. In an example, the input power for the acoustic wave generator is about 2.5 W.

In an example, an acoustic pressure applied to the cells by the acoustic wave generator is between about 0.01 MPa and 1 MPa. In an example, an acoustic pressure applied to the cells by the acoustic wave generator is between about 0.05 MPa and 1 MPa. In an example, an acoustic pressure applied to the cells by the acoustic wave generator is between about 0.05 MPa and 0.5 MPa. In an example, an acoustic pressure applied to the cells by the acoustic wave generator is between about 0.08 MPa and 0.2 MPa. In an example, an acoustic pressure applied to the cells by the acoustic wave generator is about 0.1 MPa.

In an example, the cell culture media comprises a basal media. In an example, the cell culture media does not comprise any pro-osteogenic biochemical factors such as, but not limited to, dexamethasone, ascorbic acid (or a salt thereof), β-glycerophosphate or any combination thereof. In another example, the cell culture media comprises an osteogenic media.

In an example, the disclosure provides a population of osteogenic-committed stem cells when produced by the methods disclosed herein. In an example, the population of osteogenic-committed stem cells according to the disclosure are osteoblasts.

In an example, the disclosure contemplates that the population of osteogenic-committed stem cells produced according to the methods disclosed herein are for use in the treatment of a bone disease or disorder.

In an example, the disclosure provides a method of treating a bone disease or disorder, the method comprising administering a population of osteogenic-committed stem cells produced by the methods disclosed herein.

In an example, the disclosure provides the use of a population of osteogenic-committed stem cells produced by the methods disclosed herein for the manufacture of a medicament for treating a bone disease or disorder.

In an example, the disclosure provides the use of a population of osteogenic-committed stem cells produced by the methods disclosed herein for treating a bone disease or disorder.

In an example, the population of osteogenic-committed stem cells are obtained from autologous stem cells.

In an example, the population of osteogenic-committed stem cells are obtained from allogenic stem cells.

In an example, the bone disease or disorder is selected from the group comprising osteoporosis, osteoarthritis, bone fracture, osteomyelitis, and osteonecrosis.

### DESCRIPTION OF THE FIGURES

FIG. 1—(a) Schematic representation and image of the experimental setup in which the SRBW (not to scale), generated by applying a sinusoidal electrical signal to an interdigitated transducer (IDT) photolithographically patterned onto a single crystal piezoelectric substrate (LiNbO3) at the resonant frequency (10 MHz), is coupled through a thin layer of silicon oil into a glass-bottomed culture plate containing the adherent hMSCs to promote osteogenic differentiation. (b) Viability, (c) proliferation, and, (d) extent of mineralisation of bone marrow derived hMSCs in basal (BM) and osteogenic (OM) conditions following SRBW treatment relative to the untreated control; two alternative SRBW treatment regimens were investigated: a daily single treatment regime (1×) comprising exposure of the hMSCs to the SRBW at 10 MHz for 10 mins at an input power of 2.5 W, or a daily triple treatment regime (3×) comprising successive 1× treatments 2 hours apart. The data are represented in terms of the mean value±the standard error over triplicate runs; # and † indicate statistically significant differences with p<0.01 and p<0.001, respectively.

FIG. 2—Workflows describing (a) the process for optimising the SRBW mechanostimulation parameters on bone marrow derived hMSCs, and, (b) the analysis of SRBWmediated osteogenesis. Bone marrow derived hMSCs were seeded and incubated for 48 hrs, following which the medium was replaced and the cells subjected to the SRBW stimulation for 10 mins per day over the stipulated duration. In (a), the cells were analysed by alizarin red staining on Day 14. In (b), immunofluorescence was conducted to detect RUNX2 and COL1A1 expression on Day 3, and Western blot and RT-PCR analysis was carried out on Days 3, 7, 14 and 21. Unstimulated cells in osteogenic medium and basal medium were considered as the positive and negative controls, respectively. (c) Equivalent workflow for SRBW mechanostimulation of hADSCs and hUCSCs. (d) SRBW pre-treatment of bone marrow derived hMSCs. Illustrations created using Biorender.

FIG. 3—Representative light microscopy images showing alizarin red staining of (a) unstimulated and (b) SRBW mechanostimulated hMSCs under basal conditions, and, (c) unstimulated and (d) SRBW mechanostimulated hMSCs under osteogenic conditions. The scale bars denote a length of 100 μm.

FIG. 4—Light microscopy (Eclipse TS 100, Nikon Instruments Inc., Melville, NY, USA) images of untreated and mechanostimulated bone marrow derived hMSCs (donor 1) in basal and osteogenic media. The scale bars denote a length of 100 μm.

FIG. 5—(a,b) Representative Western blot images, and, (c,d) corresponding band intensity (relative to the control) normalised against GAPDH, of proteins (osteogenic markers RUNX2 and COL1A1) isolated from the lysate of control and mechanostimulated bone marrow derived hMSCs in (a,c) basal (BM) and (b,d) osteogenic (OM) media at different post-exposure incubation periods; blot data for the replicate experiments are given in FIG. 15. The data are represented in terms of the mean value the standard error over triplicate runs; *, # and † indicate statistically significant differences with p<0.05, p<0.01 and p<0.001, respectively.

FIG. 6—Confocal immunofluorescence images (at Day 3) showing expression of RUNX2 and COL1A1 (displayed in green) in control and mechanostimulated bone marrow derived hMSCs in (a) basal (BM), and, (b) osteogenic (OM) media. Nuclei were stained with Hoechst 33342 and displayed in blue whereas actin was stained using phalloidin (ActinRed™ 555) and displayed in red. Scale bars denote a length of 50 μm. The RUNX2 nuclear/cytoplasmic ratio for these images are reported in FIG. 7. A wider field of-view image depicting RUNX2 expression in osteogenic (OM) media is also shown in FIG. 8.

FIG. 7—RUNX2 nuclear to cytoplasm (Nuc/Cyt) ratio, as quantified from immunostaining. A significant increase in the YAP Nuc/Cyt ratio can be seen in the SRBW mechanostimulated cells under both basal (BM) and osteogenic (OM) conditions with respect to that in unstimulated cells in basal media (negative control; NC) and unstimulated cells in osteogenic media (positive control; PC). The data are represented in terms of their mean value±the standard error; ‡ indicates statistically significant differences with p<0.0001 based on a t-test with 100 cells analysed per sample.

FIG. 8—Confocal immunofluorescence images (at Day 3) showing RUNX2 expression (displayed in green) in the control and mechanostimulated hMSCs in osteogenic (OM) media. Nuclei were stained with Hoechst 33342 and displayed in blue, whereas actin was stained using phalloidin (ActinRed™ 555) and displayed in red. Scale bars denote a length of 100 μm.

FIG. 9—mRNA transcriptomic profiling of (a) BMP-2, (b) RUNX2, (c) ALP, (d) COL1A1, (e) OCN and (f) OPN, showing the relative fold change obtained for the SRBW treated bone marrow derived hMSCs in basal (BM) and osteogenic (OM) media(mRNA profiles for donor 2 are shown as FIG. 16). The relative fold change in the gene expression from the SRBW-treated cells under both basal and osteogenic conditions at different time points is shown, in which the data is normalised against that of the positive control, i.e., untreated cells in osteogenic media, as is routine in relative mRNA analysis. Data for six replicates are reported, and #, t and $ indicate statistically significant differences with p<0.01, p<0.001 and p<0.0001, respectively.

FIG. 10—mRNA profiling of (a) ROCK I, (b) ROCK II, (c) Piezo1, (d) Piezo2 and (e) TRPV1 proteins in hMSCs in basal (BM) and osteogenic (OM) media at different time points in response to SRBW mechanostimulation of bone marrow derived hMSCs. The relative fold change in the gene expression from the SRBW-treated cells under both basal and osteogenic conditions at different time points is shown, in which the data is normalised against that of the positive control, i.e., untreated cells in osteogenic media, as is routine in relative mRNA analysis. Data for six replicates are reported, and *, #, † and ‡ indicate statistically significant differences with p<0.05, p<0.01, p<0.001 and p<0.0001, respectively.

FIG. 11: mRNA profiling showing (a) RUNX2 at Day 7, (b) COL1A1 at Day 7, (c) ALP at Day 7 and (d) OPN at Day 14 expression in mechanostimulated hMSCs under both basal (BM) and osteogenic (OM) conditions in the presence of a ROCK inhibitor (Y27632) or an ion channel inhibitor (ruthenium red), normalised against the unstimulated bone marrow derived hMSCs under osteogenic conditions. The data are represented as violin plot of 4 replicates; *, #, † and ‡ indicate statistically significant differences with p<0.05, p<0.01, p<0.001 and p<0.0001, respectively.

FIG. 12—Osteogenic marker (RUNX2, COL1A1 and OPN) profiling at different time points of mechanostimulated (a,b) hADSCs, (c,d) hUCSCs, and, (e,f) pre-treatment of bone marrow derived hMSCs, under (a,c,e) basal and (b,d,f) osteogenic conditions. The relative fold change in the gene expression from the SRBW-treated cells under both conditions at different time points is shown, in which the data is normalised against that of the positive control, i.e., untreated cells in osteogenic media. The data are represented as a violin plot of 6 replicates; *, #, and † indicate statistically significant differences with p<0.05, p<0.01 and p<0.001, respectively.

FIG. 13—Confocal immunofluorescence images showing RUNX2, COL1A1, COL2A1 and PPRA-γ expression (displayed in green) in the SRBW stimulated hADSCs and hUCSCs in basal media. Nuclei were stained with Hoechst 33342 and displayed in blue, whereas actin was stained using phalloidin (ActinRed™ 555) and displayed in red. The scale bars denote a length of 50 μm.

FIG. 14—(a) shows a perspective view and side view schematic of the experimental set up in which the SRBW (not to scale), generated alone a piezoelectric lithium niobate (LiNbO₃) substrate by applying an AC electric signal at the device's resonant frequency (10 MHx) to an interdigitated transducer electrode (IDT) photolithographically patterned on the substrate.

FIG. 15—Replicate Western blot data of osteogenic proteins isolated from the lysate of control and SRBW mechanostimulated hMSCs (same donor) in (a,c) basal (BM), and, (b,d) osteogenic (OM) media for different post-exposure incubation periods.

FIG. 16—mRNA profile of osteogenic markers found in control and SRBW mechanostimulated bone marrow derived hMSCs (donor 2) under (a) basal and (b) osteogenic conditions at different time points. The data are represented in terms of the mean value±the standard error; * and # indicate statistically significant differences with p<0.05 and p<0.01, respectively.

### DETAILED DESCRIPTION OF THE DISCLOSURE

### General Techniques and Selected Definitions

The term “and/or”, e.g., “X and/or Y” shall be understood to mean either “X and Y” or “X or Y” and shall be taken to provide explicit support for both meanings or for either meaning.

Reference to the singular forms “a”, “an” and “the” is also understood to imply the inclusion of plural forms unless the context dictates otherwise.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example of the disclosure unless specifically stated otherwise.

Those skilled in the art will appreciate that the disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

The present invention as described herein can be performed using, unless otherwise indicated, conventional techniques of molecular biology, recombinant DNA technology, cell biology and immunology. Such procedures are described, for example, in Sambrook, Fritsch & Maniatis, Molecular Cloning: A Laboratory Manual, Cold Spring Harbor Laboratories, New York, Second Edition (1989), whole of vols 1, 11, and III; DNA Cloning: A Practical Approach, Vols. I and II (D. N. Glover, ed., 2085), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series, Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Winsch, E., ed. (1974) Synthese von Peptiden in Houben-Weyls Metoden der Organischen Chemie (Miler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

The term “about”, as used herein when referring to a range is meant to encompass variations of ±20% or ±10%, more preferably ±5%, even more preferably ±10% from the specified amount.

As used herein, the term “treat” or “treatment” or “treating” shall be understood to refer to the medical management of a patient with the intent to cure, ameliorate, stabilize, or prevent a disease, pathological condition or disorder. This term includes active treatment, i.e. treatment directed specifically toward the improvement of a disease, pathological condition, or disorder. In addition, this term includes palliative treatment, i.e. treatment designed for the relief of symptoms rather than curing the disease, pathological condition or disorder; and supportive treatment, i.e. treatment employed to supplement another specific therapy directed towards the improvement of the associated disease, pathological condition or disorder.

As used herein, the term “subject” shall be taken to mean any subject, including a human or non-human subject. The non-human subject may include non-human primates, ungulate (bovines, porcines, ovines, caprines, equines, buffalo and bison), canine, feline, lagomorph (rabbits, hares and pikas), rodent (mouse, rat, guinea pig, hamster and gerbil), avian, and fish. In one example, the subject is a mammal. In one example, the subject is a human. In one example, the subject is a livestock animal. In one example, the subject is a companion animal.

### Osteogenic-Committed Stem Cells

As used herein, “stem cell” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells.

In an example, stem cells according to the disclosure are mesenchymal stem cells. The term “mesenchymal stem cells” includes both parent cells and their undifferentiated progeny. The term also includes mesenchymal precursor cells, multipotent stromal cells, perivascular mesenchymal precursor cells, and their undifferentiated progeny. Mesenchymal stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal stem cells are capable of differentiating into a large number of cell types including, but not limited to, adipose, osseous, cartilaginous, elastic, muscular, and fibrous connective tissues. Sources of mesenchymal stem cells include, but are not limited to, blood, skin, cord blood, muscle, fat, bone, and perichondrium. In an example, the mesenchymal stem cells are bone marrow derived mesenchymal stem cells, adipose derived mesenchymal stem cells, or umbilical cord derived mesenchymal stem cells.

In an example, the mesenchymal stem cells are autologous.

In an example, the mesenchymal stem cells are allogenic.

Methods for obtaining mesenchymal stem cells are known in the art and are described for example, in U.S. Pat. No. 5,486,359. Mesenchymal stem cells may be identified by specific cell surface markers which are identified with unique monoclonal antibodies. In an example, example, the mesenchymal stem cells express cell surface markers CD29+, CD54+, CD73+, CD90+, CD102+, CD105+, CD106+, CD166+, and/or MHC1+. A method for obtaining a cell population enriched in mesenchymal stem cells is described, for example, in U.S. Pat. No. 5,486,359.

Isolated or enriched mesenchymal stem cells can be expanded in vitro by culture. Isolated or enriched mesenchymal stem cells can be cryopreserved, thawed and subsequently expanded in vitro in a culture medium. Examples of a suitable culture media include mesenchymal stem cell growth media (MSCGM), alpha minimum essential media (αMEM), Dulbecco's Modified Eagle Medium (DMEM). Culture medium can be serum free or serum-supplemented. In an example, culture media is supplemented with 10% fetal bovine serum (FBS).

In an example, mesenchymal stem cells are expanded in an expansion culture media (e.g. MSCGM) in a humidified incubator maintained at 37° C. and 5% CO₂ until they reach a suitable confluencey, for example 80-90%. Mesenchymal stem cells can then be detached and re-seeded in a cell culture plate at a density of about 3,000 cells/cm² and incubated for 48 hrs in basal media (e.g. αMEM or DMEM). In an example, cells are passaged 3, 4, or 5 times before osteogenic differentiation.

In an example, methods of osteogenic differentiation according to the disclosure are performed in a basal culture media, examples of which are provided above. Advantageously, the inventors have discovered that the methods of osteogenic differentiation disclosed herein are particularly effective when stem cells are cultured in a basal media (i.e. in the absence of pro-oestogenic factors or other biochemical stimulation intended to facilitate osteogenesis). In another example, methods of osteogenic differentiation according to the disclosure are performed in an osteogenic culture media. In this example, cells are seeded in a cell culture plate incubated in osteogenic media.

As used herein, a “basal media” does not comprise any cell specific growth promoting agents such as any pro-osteogenic biochemical factors. The basal media may have agents which do not commit a stem cell to a certain lineage such as, fetal bovine serum or fetal calf serum (for example at 10%) or an antibiotic (for example, penicillin and/or streptomycin).

As used herein, “osteogenic differentiation” or variations thereof refers to the molecular pathways and processes undergone by stem cells as they differentiate into osteoblasts. Osteoblasts are cells that form bone tissue, synthesize and secrete bone matrix, and participate in the mineralization of bone to regulate the balance of calcium and phosphate ions in developing bone. Osteoblasts are derived from osteoprogenitor cells such as the osteogenic-committed stem cells disclosed herein.

As used herein, “osteogenic-committed stem cells” or variations thereof refers to stem cells that express known osteogenic lineage markers. In an example, osteogenic-committed stem cells express RUNX2 (runt homology domain transcription factor 2). RUNX2 is a marker for early osteogenic differentiation crucial for osteoblast development and bone formation. During osteogenic differentiation, RUNX2 is upregulated within seven days following initiation of the differentiation process, followed by downregulation during late osteogenesis to the point at which expression is completely undetectable upon maturation of the osteoblast. In an example, osteogenic-committed stem cells express RUNX2 after 3 days of exposure to acoustic wave energy according to the methods disclosed herein. In an example, osteogenic-committed stem cells express RUNX2 after 4 days, after 5 days, after 6 days, after 7 days of exposure to acoustic wave energy according to the methods disclosed herein. In an example, osteogenic-committed stem cells express RUNX2 after 5 days of exposure to acoustic wave energy according to the methods disclosed herein.

In an example, osteogenic-committed stem cells express COL1A1 (collagen type I alpha 1). In an example, osteogenic-committed stem cells express COL1A1 after 3 days of exposure to acoustic wave energy according to the methods disclosed herein. In an example, osteogenic-committed stem cells express COL1A1 after 4 days, after 5 days, after 6 days, after 7 days of exposure to acoustic wave energy according to the methods disclosed herein. In an example, osteogenic-committed stem cells express COL1A1 after 5 days of exposure to acoustic wave energy according to the methods disclosed herein. In an example, osteogenic-committed stem cells express COL1A1 after at least 5 days in the absence of acoustic wave energy following 5 days of exposure to acoustic wave energy according to the methods disclosed herein. In an example, osteogenic-committed stem cells express COL1A1 after at least 10 days in the absence of acoustic wave energy following 5 days of exposure to acoustic wave energy according to the methods disclosed herein. In an example, osteogenic-committed stem cells express COL1A1 after at least 16 days in the absence of acoustic wave energy following 5 days of exposure to acoustic wave energy according to the methods disclosed herein.

In an example, osteogenic-committed stem cells express bone morphogenetic protein-2 (BMP-2). In an example, osteogenic-committed stem cells express BMP-2 after 3 days of exposure to acoustic wave energy according to the methods disclosed herein. In an example, osteogenic-committed stem cells express BMP-2 after 4 days, after 5 days, after 6 days, after 7 days of exposure to acoustic wave energy according to the methods disclosed herein. In an example, osteogenic-committed stem cells express BMP-2 after 5 days of exposure to acoustic wave energy according to the methods disclosed herein.

In an example, osteogenic-committed stem cells express alkaline phosphatase (ALP). ALP is an indicator of mineralisation during the early stages of osteoblast commitment. In an example, osteogenic-committed stem cells express ALP after 3 days of exposure to acoustic wave energy according to the methods disclosed herein. In an example, osteogenic-committed stem cells express ALP after 4 days, after 5 days, after 6 days, after 7 days of exposure to acoustic wave energy according to the methods disclosed herein. In an example, osteogenic-committed stem cells express ALP after 5 days of exposure to acoustic wave energy according to the methods disclosed herein. In an example, osteogenic-committed stem cells express ALP after at least 5 days in the absence of acoustic wave energy following 5 days of exposure to acoustic wave energy according to the methods disclosed herein. In an example, osteogenic-committed stem cells express ALP after at least 10 days in the absence of acoustic wave energy following 5 days of exposure to acoustic wave energy according to the methods disclosed herein. In an example, osteogenic-committed stem cells express ALP after at least 16 days in the absence of acoustic wave energy following 5 days of exposure to acoustic wave energy according to the methods disclosed herein.

In an example, osteogenic-committed stem cells express osteocalcin (OCN). OCN is a late stage osteogenic marker and is bone-specific extracellular matrix protein secreted by osteoblasts whose expression provides a measure of its mineralisation activity. In an example, osteogenic-committed stem cells express OCN after 3 days of exposure to acoustic wave energy according to the methods disclosed herein. In an example, osteogenic-committed stem cells express OCN after 4 days, after 5 days, after 6 days, after 7 days of exposure to acoustic wave energy according to the methods disclosed herein. In an example, osteogenic-committed stem cells express OCN after 5 days of exposure to acoustic wave energy according to the methods disclosed herein. In an example, osteogenic-committed stem cells express OCN after at least 5 days in the absence of acoustic wave energy following 5 days of exposure to acoustic wave energy according to the methods disclosed herein. In an example, osteogenic-committed stem cells express OCN after at least 10 days in the absence of acoustic wave energy following 5 days of exposure to acoustic wave energy according to the methods disclosed herein. In an example, osteogenic-committed stem cells express OCN after at least 16 days in the absence of acoustic wave energy following 5 days of exposure to acoustic wave energy according to the methods disclosed herein.

In an example, osteogenic-committed stem cells express osteopontin (OPN). OPN is a late stage osteogenic marker. Enhancement in OPN expression has been implicated in osteoblastic bone formation under mechanical stress. In an example, osteogenic-committed stem cells express OPN after at least 5 days in the absence of acoustic wave energy following 5 days of exposure to acoustic wave energy according to the methods disclosed herein. In an example, osteogenic-committed stem cells express OPN after at least 10 days in the absence of acoustic wave energy following 5 days of exposure to acoustic wave energy according to the methods disclosed herein. In an example, osteogenic-committed stem cells express OPN after at least 16 days in the absence of acoustic wave energy following 5 days of exposure to acoustic wave energy according to the methods disclosed herein.

Methods of measuring expression of osteogenic markers disclosed herein are known in the art. In an example, protein expression of osteogenic markers in cells is measured by Western blot. In an example, protein expression of osteogenic markers in cells is measured by immunofluorescence. For example, a cell sample is harvested and contacted with an antibody against osteogenic markers. Using a detectable label (conjugated to the anti-osteogenic marker antibody or one that binds to the anti-osteogenic marker antibody), protein expression can be imaged and quantified.

In another example, gene expression (e.g. RNA expression) of osteogenic markers in cells is measured by real-time quantitative polymerase chain reaction (RT-qPCR). For example, a cell sample is harvested and treated to obtain total RNA content from which cDNA is generated. PCR is then performed using primers targeting the gene/s of interest.

In an example, the osteogenic marker is expressed at least at about a 2 fold, or at least at about a 3 fold, or at least at about a 4 fold, or about a 2 fold to about a 5 fold, or about a 2 fold to about a 5 fold, higher level than if the cells were cultured under the same conditions by exposed to a frequency of 1 MHz or less, or 3 MHz or less.

### Acoustic Wave Energy

An acoustic wave (also known as “sound wave”), is a mechanical wave generated by a vibrating surface or object. Acoustic waves mainly propagate through an elastic media as longitudinal (pressure; vibration displacement parallel to the direction of wave propagation) waves that involve the compression and rarefaction of the molecules in that medium. As it is possible to support vibrations in other directions in solids, transverse (shear) waves can also exist where the vibration displacement is transverse to the direction of wave propagation.

The acoustic wavelength λ is related to the frequency f by the speed at which the sound wave propagates through the medium c, which is dependent on its density and elasticity. Given this relationship and the broad spectrum of sound frequencies—ranging from infrasound (<20 Hz), audible (20 Hz-20 kHz), ultrasound (20 kHz-1 GHz) to hypersound (>1 GHz)—together with the different configurations of acoustic wave generation devices, different wave modes—i.e., the different ways acoustic waves can propagate through the media—can arise.

Types of acoustic waves include bulk acoustic waves (BAWs), surface acoustic waves (SAWs; also known as Rayleigh waves) and surface reflected bulk waves (SRBWs; also known as pseudo-SAWs) which are a hybrid surface and bulk wave. Bulk acoustic waves do not only take the form of longitudinal (pressure) or transverse (waves). Thin solid sheets with thicknesses h<λ (or h/λ<1), for example, can support plate waves that propagate parallel to its surface and through the thickness of the material. If the plate is infinitely wide, only a thickness mode exists, whereas a plate with finite width gives rise to symmetric (extensional) or asymmetric (flexural) Lamb waves that comprise both thickness and width modes. SAWs in contrast, occur in piezoelectric substrates whose thicknesses are much greater than the acoustic wavelength, i.e., h>λ or h/λ>1, and supports a combination of both longitudinal and transverse waves. SRBWs on the other hand, can also exist in the intermediate transition regime where h≈λ or h/λ≈1.

The coupling of sound waves to laboratory cell cultureware can be achieved with the use of an apparatus comprising an acoustic wave generator or transducer as disclosed herein. At low frequencies in the infrasound and audible range, longitudinal bulk acoustic waves can be induced in a culture chamber by coupling the piston-like vibration generated with a conventional sound transducer akin to that found in loudspeakers. To generate BAWs at ultrasound frequencies up to several MHz, piezoceramic transducers are typically used, on which electrode pads are patterned (alternatively, higher frequency (MHz) BAWs can also be generated on piezoelectric substrates). For SAWs and SRBWs, a chipscale piezoelectric substrate is employed, on which interdigitated transducers (IDTs)—electrodes patterned in an interleaved pattern—are photolithographically patterned, whose gap and spacing determines the resonant frequency of the device and hence the wavelength and frequency of the SAW or SRBW that propagates along the substrate. Whether a SRBW or SAW is generated depends on this resonant frequency and hence wavelength relative to the substrate thickness.

In an example, the acoustic wave energy is provided as surface acoustic waves (SAWs).

In an example the acoustic wave energy is provided as surface reflected bulk waves (SRBWs).

In an example, the acoustic wave energy is provided at a high frequency, i.e. greater than 1 kHz.

In an example, the acoustic wave energy is provided to the stem cells at a frequency greater than 1 MHz. In an example, the acoustic wave energy is provided to the stem cells at a frequency of at least about 2 MHz. In an example, the acoustic wave energy is provided to the stem cells at a frequency greater than 3 MHz. In an example, the acoustic wave energy is provided to the stem cells at a frequency between about 1 MHz and about 1 GHz. In an example, the acoustic wave energy is provided to the stem cells at a frequency between greater than 1 MHz and about 1 GHz. In an example, the acoustic wave energy is provided to the stem cells at a frequency between greater than 3 MHz and about 1 GHz. In an example, the acoustic wave energy is provided to the stem cells at a frequency between about 3 MHz and about 50 MHz. In an example, the acoustic wave energy is provided to the stem cells at a frequency between about 4 MHz and about 50 MHz. In an example, the acoustic wave energy is provided to the stem cells at a frequency between about 5 MHz and about 35 MHz. In an example, the acoustic wave energy is provided to the stem cells at a frequency between about 25 MHz and about 35 MHz. In an example, the acoustic wave energy is provided to the stem cells at a frequency between about 5 MHz and about 15 MHz. In an example, the acoustic wave energy is provided to the stem cells at a frequency between about 8 MHz and about 12 MHz. In an example, the acoustic wave energy is provided to the stem cells at a frequency of about 10 MHz.

The acoustic energy received by the cells is dependent on the input power of the acoustic wave generator as well as the dimensions, materials and configuration of the apparatus including the acoustic wave generator, the receptacle and the properties and quantity of the culture medium.

In some embodiments, the apparatus may be configured such that the acoustic energy applied to the cells by the acoustic wave generator is in the range of about 0.1 W to about 10 W, about 1 W to about 10 W, about 1 W to about 5 W, about 0.001 W to about 0.1 W, 1 mW to 50 mW, 1 mW to 10 mW, 10 mW to 100 mW, 20 mW to 80 mW, 30 mW to 60 mW, or less than 100 mW, less than 80 mW, less than 50 mW, less than 30 mW, or about 50 mW. The acoustic pressure applied to the cells by the acoustic wave generator may be in the range of 0.01 MPa to 1 MPa, for example. In an example, the acoustic pressure applied to the cells by the acoustic wave generator is about 0.1 MPa. In an example, an acoustic pressure applied to the endothelial cells by the acoustic wave generator is between about 0.05 MPa and about 0.5 MPa. In an example, an acoustic pressure applied to the endothelial cells by the acoustic wave generator is between about 0.08 MPa and about 0.2 MPa.

The inventors have identified that exposing stem cells to acoustic wave energy intermittently for a short duration is sufficient to induce differentiation towards an osteogenic lineage. Accordingly, in an example, the stem cells are exposed to acoustic wave energy for a period between about 30 seconds to about 20 minutes. In an example, endothelial the cells are exposed to acoustic wave energy for a period between about 5 minutes to about 15 minutes. In an example, endothelial the cells are exposed to acoustic wave energy for a period of about 10 minutes.

Methods of the present disclosure also encompass exposing the population of cells to a period of acoustic wave energy followed by incubation in the absence of acoustic stimulation. In an example, the population of stem cells is exposed to one or more periods of acoustic wave energy followed by incubation in the absence of acoustic stimulation. In an example, the population of stem cells are exposed to acoustic wave energy for a short duration (e.g. about 10 minutes) followed by incubation in the absence of acoustic stimulation for between about 1 hour and about 24 hours. In an example, the incubation in the absence of acoustic stimulation is between about 6 and 20 hours. In an example, the incubation in the absence of acoustic stimulation is between about 8 and 12 hours.

In an example, the cells are exposed to acoustic wave energy for a short duration period once, twice, 3 times, 4 times, 5 times, 6 times, 7 times, 8 times, 9 times, or 10 times per day. In an example the cells are exposed to acoustic wave energy for a short duration period once per day. In an example, the cells are exposed to acoustic wave energy for a short duration period for at least 3 days, for at least 4 days, or for at least 5 days. In an example, the cells are exposed to acoustic wave energy for 5 days.

### Apparatus

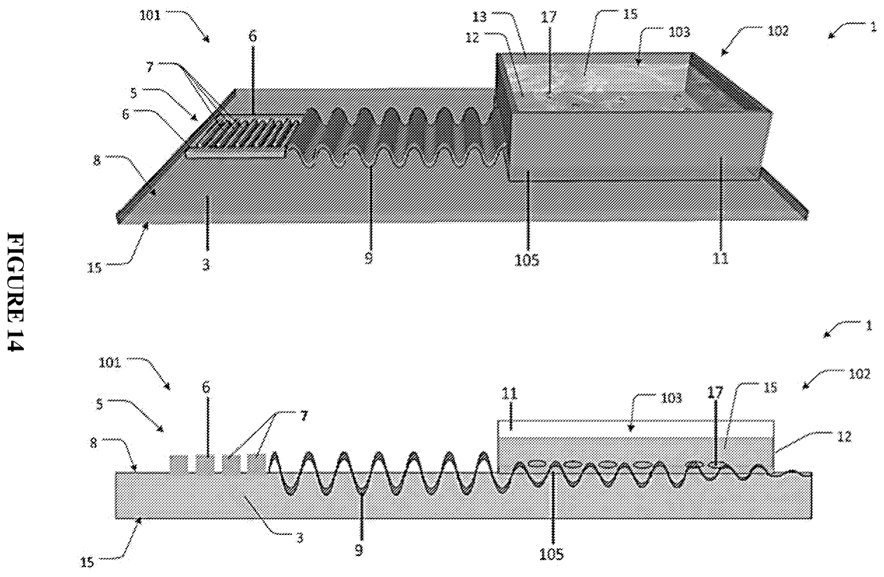

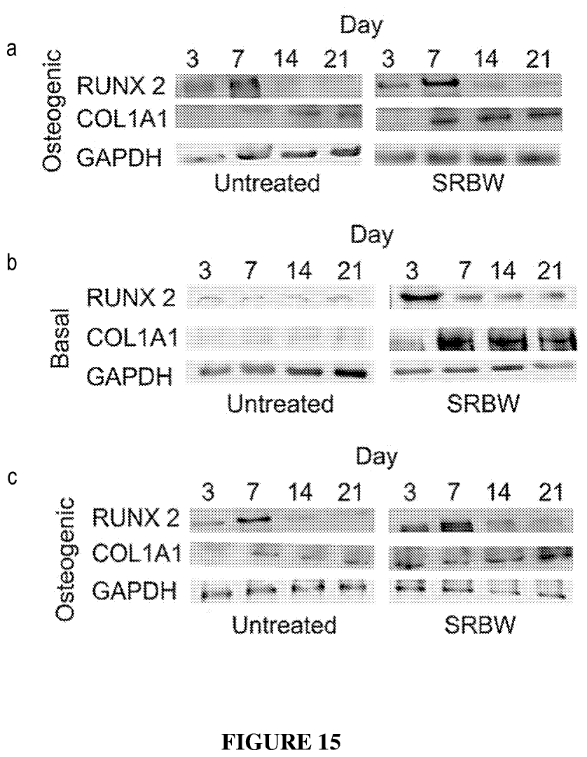

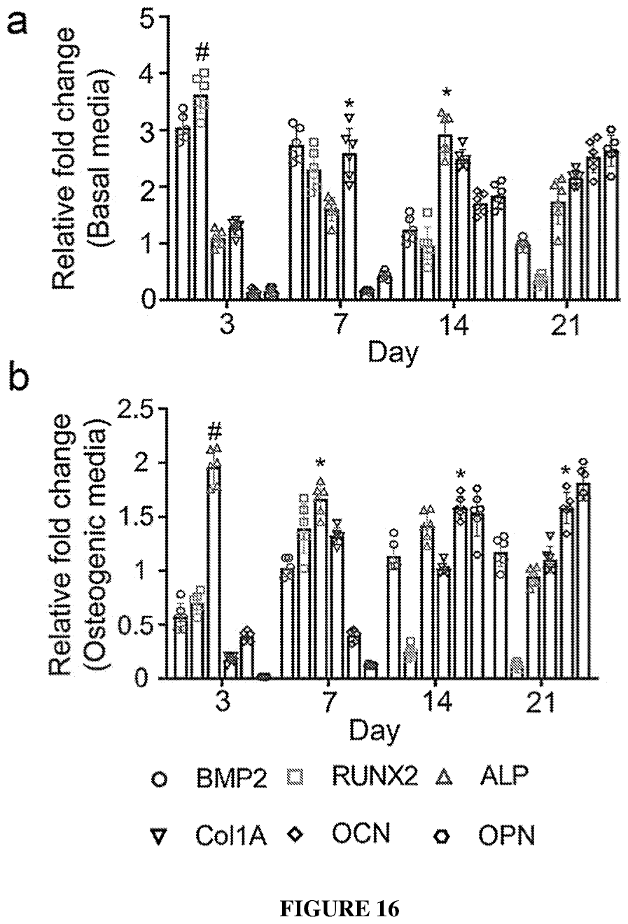

Referring to FIG. 14, an apparatus 1 for exposing a population of stem cells to acoustic energy (i.e., acoustic waves or vibrations) is shown, according to some embodiments.

The apparatus 1 comprises an acoustic wave generator 101 configured to generate acoustic energy at a selected power and frequency; and a receptacle 102 for accommodating a population of cells. The receptacle 102 is configured to receive acoustic energy generated by the acoustic wave generator 101.

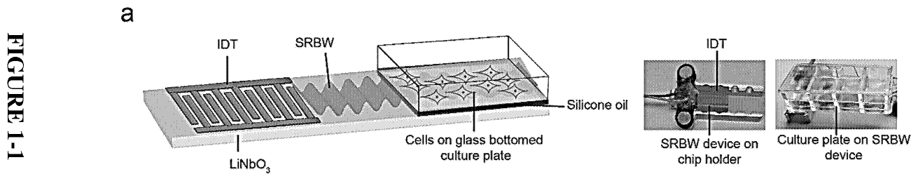

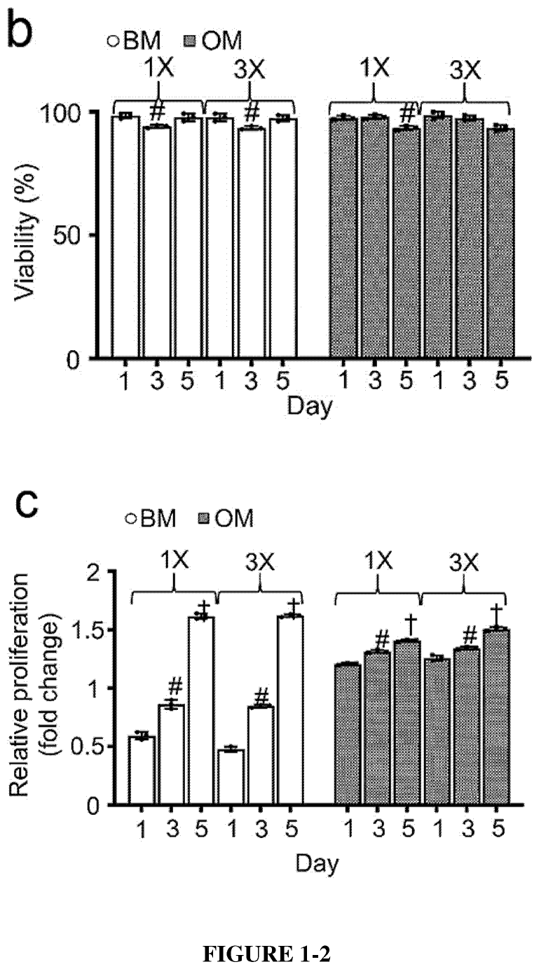

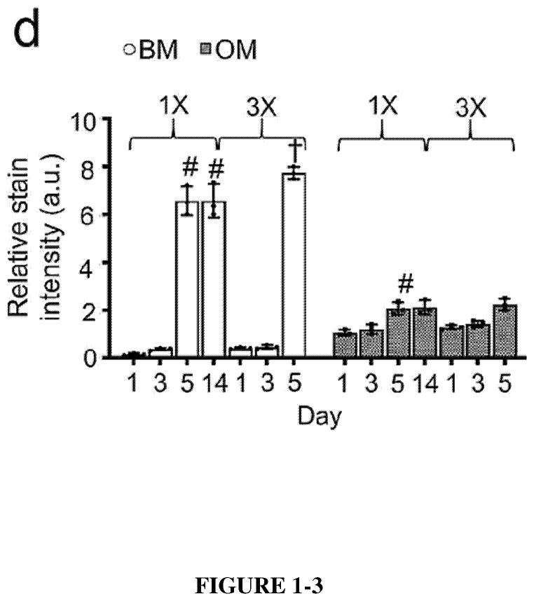

The acoustic wave generator 101 shown in FIG. 1a comprises a piezoelectric element. However, in other embodiments, the acoustic wave generator 101 may comprise any other suitable device for generating acoustic energy or vibrations, including speakers, vibrators or other electromechanical devices.

The receptacle 102 may define a reservoir 103 configured to accommodate the population of cells in a cell medium. For example, the reservoir 103 may define a well, or in some embodiments, the reservoir may define a channel allowing the cells and culture medium to flow into and/or out of the reservoir.

The receptacle 102 may be coupled to the acoustic wave generator 101. For example, the receptacle 102 may be coupled to the acoustic wave generator 101 with a coupling material 105 to facilitate transmission of the acoustic energy from the acoustic wave generator 101 to the receptacle 102. For example, the coupling material 105 may comprise a fluid couplant, such as silicone oil.

In some embodiments, the receptacle 102 may be fixed to the acoustic wave generator 101. For example by adhesive bonding or by mechanical fastening.

In some embodiments, the receptacle 102 may be separate from the acoustic wave generator 101. For example, the receptacle 102 may not be in direct contact with the acoustic wave generator 101 during operation. The acoustic energy may be transmitted from the acoustic wave generator 101 to the receptacle 102 via a transmission medium.

The apparatus 1 shown in FIG. 1a includes a piezoelectric substrate 3, for example, lithium niobate (LiNbO₃), defining a working surface 8 on which electrodes 6 of an interdigitated transducer (IDT) 5 are photolithographically patterned. The width of and gaps between the IDT fingers 7 of the electrodes 6 determine the resonant wavelength and resonant frequency of the acoustic wave generator 101.

Applying an alternating electrical signal to the IDT electrodes 6 at this resonant frequency with the aid of a signal generator and amplifier (not shown) then generates surface acoustic waves (SAW) 9 that propagate as Rayleigh waves along the working surface 8 of the substrate 3 upon which the IDT electrodes 6 are positioned. In addition to the SAW 9, surface reflected bulk waves (SRBW) can also propagate internally within the substrate 3 between the working surface 8, and an adjacent opposing surface 15 of the substrate 3. The SRBW is internally reflected between the working surface 8 and the opposing surface 15 and preferably also provides acoustic wave energy to the receptacle 102. The propagation of the SRBW may be enhanced by configuring the substrate 3 so that it has a thickness that is approximately equal to the SAW wavelength. Further description of SRBWs can be found in International Publication No. WO2016/179664 (RMIT University).

The receptacle 102 of the apparatus 1 of FIG. 14 is shown in the form of a well plate 11, comprising a base 12 and side walls 13 made from glass or other acoustically transmitting materials such as acrylic. The receptacle 102 is disposed on the working surface 8 of the substrate 3. The receptacle 102 defines multiple wells each configured to accommodate a population of cells 17 in cell media 15. Alternatively, the receptacle 102 may comprise one or more petri dishes, transwell culture plates, microarray plates, cell flack, or other standard laboratory items for cell culture made from glass or other suitable materials. Additionally, the receptacle 102 may also comprise a fluid channel or conduit as part of a flow through system.

In some embodiments, the receptacle 102 may be integrally formed with the acoustic wave generator 101. For example, the receptacle 102 may comprise a portion of the acoustic wave generator 101, such as a portion of the substrate 3. The reservoir 103 may be defined by a recess in the working surface 8, for example.

It is also envisaged that a receptacle 102 having side walls only and no base wall could be used so that the cells and media 15 can be in direct contact with (i.e., directly coupled to) the working surface 8.

The receptacle 102 may be positioned on the work surface 8 to transmit the acoustic wave energy of the SAW 9 and preferably SRBW to the accommodated cells 17. A thin layer of silicone oil (or another fluid couplant, including water, glycerine, or other acoustic transmitting materials including gels and tapes) may be placed between the working surface 8 and base wall 12 of the well plate 11 to facilitate the coupling between the acoustic wave generator 101 and the receptacle 102, and to facilitate the transmission of the acoustic wave energy into the wells. The silicone oil may also mitigate or reduce any acoustic impedance mismatch.

In some embodiments, the acoustic wave generator 101 and substrate 3 may not be in direct contact with the receptacle 102, and may be configured to be entirely separate from the receptacle 102. For example, the acoustic wave generator 101 may be arranged to transmit acoustic energy to the cells via a transmission medium or fluid, such as a gas or liquid. The transmission medium may comprise air and/or a liquid cell medium or culture medium accommodating the cells.

In some embodiments, the substrate 3 of the acoustic wave generator 101 may be configured such that a plane of the substrate 3 is substantially perpendicular relative to a bottom plane of the receptacle. For example, the substrate 3 may be arranged substantially vertically relative to the substantially horizontal receptacle as shown in FIG. 1(b).

In an example, at least part of the acoustic wave generator is arranged in direct contact with the culture medium

In an example, the input power for the acoustic wave generator is in the range of about 1 W to about 10 W. In an example, the input power for the acoustic wave generator is in the range of about 2 W to about 5 W. In an example, the input power for the acoustic wave generator is in the range of about 2 W to about 3 W. In an example, the input power for the acoustic wave generator is about 2.5 W.

In an embodiment, the duty cycle is between 20% and 100%. In an embodiment, the duty cycle is between above 50% and 100%. In an embodiment, the duty cycle is between 75% and 100%. In an embodiment, the duty cycle is 100%.

### Compositions

Osteogenic-committed stem cells produced by the methods of the present disclosure may be provided in the form of a composition comprising a pharmaceutically acceptable carrier and/or excipient. The choice of excipient or other elements of the composition can be adapted in accordance with the route and device used for administration.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Administration to a subject (e.g. human) is preferably by injection. The administration route may be intramuscular or intravascular (e.g. intravenous), intra-articular, intracerebral, subcutaneous or transdermal. A physician will be able to determine the required route of administration for each particular patient.

A composition of the invention may be formulated for parenteral, intramuscular, intracerebral, intravascular (including intravenous), intra-articular, subcutaneous or transdermal administration.

Suitable carriers for the present disclosure include those conventionally used, e.g. water, saline, aqueous dextrose, lactose, Ringer's solution a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. Such a media composition does not induce any adverse effects in a subject to whom it is administered. Exemplary carriers and excipients do not adversely affect the viability of a cell and/or the ability of a cell to treat or prevent disease.

In an example, compositions of the disclosure comprise between 10×10⁶ cells and 35×10⁶ cells. In another example, the composition comprises between 20×10⁶ cells and 30×10⁶ cells. In other examples, the composition comprises at least 100×10⁶ cells. In another example, the composition comprises between 50×10⁶ cells and 500×10⁶ cells. In other examples, compositions of the disclosure comprise 150 million cells.

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of osteogenic-committed stem cells and/or progeny thereof and/or soluble factor derived therefrom. For example, the composition comprises about 1×10⁵ cells to about 1×10⁹ cells or about 1.25×10³ cells to about 1.25×10⁷ cells/kg (80 kg subject). The exact amount of cells to be administered is dependent upon a variety of factors, including the age, weight, and sex of the subject, and the extent and severity of the disorder being treated.

### Methods of Treatment

The present disclosure also provides medical uses and methods for treatment in vitro and in vivo of the osteogenic-committed stem cells produced herein. In one example, the osteogenic-committed stem cells are used in the treatment of a bone disease or disorder. In a further example, the bone disease or disorder includes degenerative bone conditions, disorders resulting in reduced bone repair ability, bone loss and bone defects. In a further example, the bone disease or disorder is one which can be treated by bone graft, including auto-grafts, allo-grafts and xeno-grafts, or by bone regeneration. In a further example, the bone disease or disorder is selected from the group consisting of osteoporosis, osteomalacia, osteomyelitis, bone fracture, osteonecrosis and atrophic non-union.

Osteoporosis is a metabolic bone disorder in which bone mineralisation and formation is slower than bone degradation and resorption, resulting in brittle or weak bones. Osteomalacia is also characterised by decreased mineralisation and faster resorption than formation, but results in soft bones, rather than the brittle bones of osteoporosis. Osteomyelitis is infection of the bone, caused by, for example, pyogenic bacteria, fungi or mycobacteria and can be chronic or acute. Bone fracture is a break or damage to the bone, and can be caused by or worsened by osteoporosis, osteomyelitis and osteonecrosis. Osteonecrosis is the death of bone tissue as a result of loss of blood flow to bone cells and can but does not necessarily follow injury to the bone. Atrophic non-union is the delayed or failed healing of a bone fracture after injury which may be caused by reduced blood flow, similar to osteonecrosis, or by other factors including inadequate fracture stabilisation, infection, environmental and genetic factors.

In example, methods for treatment of the bone disease or disorder comprises administering to a subject a composition comprising osteogenic-committed stem cells produced by the methods disclosed herein. In an example, the subject is treated with an a composition comprising allogenic osteogenic-committed stem cells. In another example, the subject is treated with an a composition comprising autologous osteogenic-committed stem cells.

Subjects can be treated via administration compositions comprising osteogenic-committed stem cells as disclosed herein. In an example, the administration route is be intramuscular or intravascular (e.g. intravenous), intra-articular, intracerebral, subcutaneous or transdermal. A physician will be able to determine the required route of administration for each particular patient.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Example 1: Methods and Materials

Sodium chloride, methanol, ethanol, isopropanol, liquid ethane, ammonium hydroxide, acetic acid, RNase-free water, nuclease-free water, glycerol, glycerine, non-fat skimmed milk, silicon oil, dimethylsulphoxide (DMSO), β-mercaptoethanol, Tween® 20, sodium dodecyl sulphate (SDS), Trizma® (Tris) base, phosphate buffered saline (PBS), amiloride hydrochloride (HCl), Tris-HCl, chloroform, ammonium persulphate (APS), bis-acrylamide, Gibco penicillin-streptomycin (PS), acetylthiocholine, ethylenediaminetetraacetic acid (EDTA), trypsin-EDTA, formaldehyde, Triton™ X-100, bovine serum albumin (BSA), fetal bovine serum (FBS), Dulbecco's Modified Eagle Medium (D-MEM), a minimal essential medium (α-MEM), Dulbecco's phosphate buffered saline (D-PBS), bromophenolblue, radioimmunoprecipitation (RIPA) assay buffer, biotinylated protein ladder, Hoechst 33342, ActinRed™ 555, Trypan Blue solution, ruthenium red, alizarin red, ammonium hydroxide, 3-(4,5-dimethylthiazol-2-yl)-2,5-diphenyltetrazolium bromide (MTT), trypan blue, ruthenium red, bicinchoninic acid (BCA) protein assay kit, LunaScript® RT Super-Mix kit, Luna® Universal qPCR Master Mix, TRiZOL™ reagent, Pierce™ ECL Western blotting detection reagent, nitrocellulose membrane (0.45 m), protease inhibitor cocktail tablets, polyacrylamide gel, T25 cell culture flasks, Nunc™ 8-well plate, and, Nunc™ Lab-Tek™ II Chamber Slide and Chambered Coverglass were acquired from Thermo Fischer Scientific Pty. Ltd. (Scoresby, VIC, Australia). The ROCK inhibitor (Y27632) was purchased from Sigma-Aldrich Pty. Ltd. (Castle Hill, NSW, Australia). Human mesenchymal stem cells, mesenchymal stem cell basal medium (MSCBM™), mesenchymal stem cell growth medium (MSCGM™) and osteogenic medium (containing dexamethasone, ascorbate, L-glutamine, β-glycerophosphate, penicillin-streptomycin and mesenchymal cell growth supplement) were procured from Lonza Pty. Ltd. (Mount Waverley, VIC, Australia). Human adipose derived stem cells (hADSCs) and human umbilical cord blood derived stem cells (hUCSCs) together with the Mesenchymal Stem Cell Growth Kit for Adipose and Umbilical-Derived MSCs-Low Serum were procured from American Type Culture Collection (Manassas, VA, USA). Anti-GAPDH mouse antibody, anti-RUNX2 rabbit antibody, anti-COL1A1 rabbit antibody, anti-PPRA-γ rabbit antibody, anti-mouse horse radish peroxidase (HRP) conjugated antibody, anti-biotin HRP linked antibody, and anti-rabbit HRP-conjugated antibody were obtained from Cell Signalling Technology Inc. (Danvers, MA, USA), whereas anti-COL2A1 mouse antibody was obtained from St. Vincent's Hospital, Melbourne, VIC, Australia. (Y27632) was purchased from Sigma-Aldrich Pty. Ltd. (Castle Hill, NSW, Australia).

The following primers used for RT-qPCR analysis were acquired from Integrated DNA Technologies Inc. (Coralville, IA, USA):

β-actin (forward):
(SEQ ID NO: 1)
5′-TGACGGGGTCACCCACACTGTGCCCAT-3′,
β-actin (reverse):
(SEQ ID NO: 2)
5′-CTAGAAGCATTTGCGGTGGACGATGGAGGG-3′,
ROCK I (forward):
(SEQ ID NO: 3)
5′-ACCTGTAACCCAAGGAGATGTG-3′,
ROCK I (reverse):
(SEQ ID NO: 4)
5′-CACAATTGGCAGGAAAGTGG-3′,
ROCK II (forward):
(SEQ ID NO: 5)
5′-AAGTGGGTTAGTCGGTTG-3′,
ROCK II (reverse):
(SEQ ID NO: 6)
5′-GGCAGTTAGCTAGGTTTG-3′,
Piezo 1 (forward):
(SEQ ID NO: 7)
5′-TCGCTGGTCTACCTGCTCTT-3′,
Piezo 1 (reverse):
(SEQ ID NO: 8)
5′-GGCCTGTGTGACCTTGG-3′,
Piezo 2 (forward):
(SEQ ID NO: 9)
5′-CCCGGAGTTTGAAAATGAAG-3′,
Piezo 2 (reverse):
(SEQ ID NO: 10)
5′-CAGTGCCTCTTCTGAATCAATTT-3′,
TRPV1 (forward):
(SEQ ID NO: 11)
5′-AGAGTCACGCTGGCAACC-3′,
TRPV1 (reverse):
(SEQ ID NO: 12)
5′-GGCAGAGACTCTCCATCACAC-3′,
OPN (forward):
5′-AGCTGGATGACCAGAGTGCT-3′,
(SEQ ID NO: 13)
OPN (reverse):
5′-TGAAATTCATGGCTGTGGAA-3′,
(SEQ ID NO: 14)
ALP (forward):
5′-ATGAAGGAAAAGCCAAGCAG-3′,
(SEQ ID NO: 15)
ALP (reverse):
5′-CCACCAAATGTGAAGACGTG-3′,
(SEQ ID NO: 16)
COLIA1 (forward):
(SEQ ID NO: 17)
5′-ACATGTTCAGCTTTGTGGACC-3′,
COL1A1 (reverse):
(SEQ ID NO: 18)
5′-TGATTGGTGGGATGTCTTCGT-3′,
BMP-2 (forward):
(SEQ ID NO: 19)
5′-ATGGATTCGTGGTGGAAGTG-3′,
BMP-2 (reverse):
(SEQ ID NO: 20)
5′-GTGGAGTTCAGATGATCAGCG-3′,
OCN (forward):
(SEQ ID NO: 21)
5′-GACTGTGACGAGTTGGCTGA-3′,
OCN (reverse):
(SEQ ID NO: 22)
5′-CTGGAGAGGAGCAGAACTGG-3′,
RUNX2 (forward):
(SEQ ID NO: 23)
5′-AAGTGCGGTGCAAACTTTCTT-3′,
RUNX2 (reverse):
(SEQ ID NO: 24)
5′-TCTCGGTGGCTGGTAGTGA-3′,
PPIA (forward):
(SEQ ID NO: 25)
5′-CGGGTCCTGGCATCTTGT-3′,
PPIA (reverse):
(SEQ ID NO: 26)
5′-CAGTCTTGGCAGTGCAGATGA-3′.

### Device Fabrication

The SRBW devices, shown in FIG. 1, comprised 40 alternating fingers of 11-mm-wide and 66-nm-thick straight interdigitated aluminium transducer (IDT) electrodes on 500-μm-thick 127.86° Y-X rotated lithium niobate (LiNbO3) single-crystal piezoelectric substrates (Roditi Ltd., London, UK) in a basic full-width interleaved configuration. Sputter deposition and standard ultraviolet (UV) photolithography were used to pattern the IDTs atop a 33-nm-thick chromium adhesion layer. The device's resonant frequency f was set at 10 MHz by the width and the gap of the IDT fingers (λ/4), which specifies the SRBW wavelength, =398 am. A signal generator (SML01, Rhode & Schwarz Pty. Ltd., North Ryde, NSW, Australia) and amplifier (10W1000C, Amplifier Research, Souderton, PA, USA) was used to generate the SRBW by applying an alternating electrical signal to the IDTs at the resonant frequency. The acoustic wave energy from the device was relayed to the cells that were adherent to the glass-bottomed (0.15 mm thickness) square-well chamber slide through a thin layer of silicon oil with viscosity 45-55 cP and density 0.963 g/ml at 25° C.

Accounting for transmission losses through the fluid coupling layer and cultureware, the characteristic displacement velocity U, on the order 10-2 m/s associated with the ω=10 MHz vibrational excitation, gives rise to surface accelerations on the order a≈Uω≈10⁵ m/s2 or 10⁴ g's. This large magnitude is not surprising given that the surface acceleration on the piezoelectric lithium niobate (LiNbO3) substrate, on which the SRBW is generated, itself is typically around 10⁶ g's (Rezk et al., 2021). The significantly larger acceleration at higher excitation frequencies is also expected given that a~ω2: before considering any transmission losses, the acceleration associated with 10 MHz oscillations of the same displacement amplitude (as measured using laser Doppler vibrometry) to their 1 kHz counterpart increases by a factor of 108. Consequently, the cells experience considerably larger periodic pressures (on the order 0.1 MPa, consistent with that observed in other high frequency studies (Zhang et al., 2017)) and hence apparent gravity, which has been known to promote osteogenesis (Pemberton et al., 2015; Holick 1998; Thellin et al., 1999; Garman et al., 2007).

### Cell Culture and Mechanostimulation

Human bone marrow derived mesenchymal stem cells (hMSCs; two donors), adipose derived stem cells (hADSC; one donor) and umbilical cord blood derived stem cells (hUCSC; one donor) were expanded in their respective media (Table 1/Table 3.1) in a humidified incubator maintained at 37° C. and 5% CO₂. The cells were grown in a standard T25 flask until they reached 80-90% confluency, following which they were detached using 0.05% trypsin-EDTA and reseeded in 8-well plates at a density of 3,000 cells/cm2 and incubated for 48 hrs in basal media (BM; α-MEM with FBS and PS), ensuring proper adhesion. Cells at passage 3, 4 and 5 were used for the study. The media was then replaced with fresh basal media and another set of cells were incubated in osteogenic media (OM). The cells in the 8-well plate were then irradiated with the 10 MHz SRBW at 2.5 W input power for 10 mins, which constituted a single treatment regimen (1×). Following cessation of the vibrational excitation, the cells were incubated overnight at 37° C. Unless otherwise stated, the standard treatment (1×) and subsequent incubation was carried out each day over the next four days for various time point studies. The respective media was replaced every two days. Control samples constituted cells seeded in the respective media at the same density and incubated over the same time period, but devoid of any vibrational excitation. The positive control consisted of these untreated cells in osteogenic media whereas the negative control consisted of untreated cells in basal media. Replicate experiments were conducted with cells from the same donor in different 8-well plates.

For the reseeding experiments, hMSCs under basal conditions were exposed to the SRBW for 5 days. The stimulated cells were then trypsinised and reseeded onto a 48-well plate on Day 6. The cells were allowed to grow in basal media until Day 21.

For the inhibitor studies, the hMSCs were seeded at a density of 3,000 cells per well. After initial 48 hrs of cell growth in their respective media, the cells were then subjected to 10 μM of the ROCK inhibitor (Y27632). For piezo channel inhibition, the cells were exposed to 10 μM of cationic ion channel blocker (ruthenium red) for 20 mins prior to SRBW mechanostimulation and 10 mins after the treatment media was replaced. Similar to ROCK inhibition, the cells were treated with ruthenium red.

TABLE 1
Culture media used for the stem cells from
various tissue sources at different
stages in the experiments.

| Stem | Expansion | | Differentiation |
|---|---|---|---|
| Cell | Media | Basal Media | Media |
| BMD- | MSCGM | α-MEM | Osteogenic media |
| hMSC | (Lonza) | (10% FBS, 1% PS) | (Lonza) |
| hADSC | MSCGM | D-MEM | Osteogenic media |
| | (ATCC) | (10% FBS, 1% PS) | (Lonza) |
| hUCSC | MSCGM | α-MEM | Osteogenic media |
| | (ATCC) | (10% FBS, 1% PS) | (Lonza) |

### Cell Viability

The viability of the hMSCs following their exposure to the SRBW irradiation was assessed using an MTT proliferation assay in which the treated cells were washed with PBS immediately after collecting the spent media, following which the MTT solution at a final concentration of 0.5 mg/ml in serum-free medium was added to each well and incubated for 3 hrs. The formazan crystals dissolved in DMSO was measured at 570 nm using a spectrophotometric plate reader (CLARIOstar®, BMG LabTech, Mornington, VIC, Australia) and normalised with respect to the absorbance of the control containing cells at the same concentration that were not exposed to the SRBW irradiation but incubated for the same time period. The viability of the SRBW-treated cells after various incubation periods was analysed. The assay was carried out 24 hours after the SRBW treatment. For proliferative studies, the increase in viable cells was measured using the MTT assay, 7 days after the SRBW treatment.

### Alizarin Red Staining

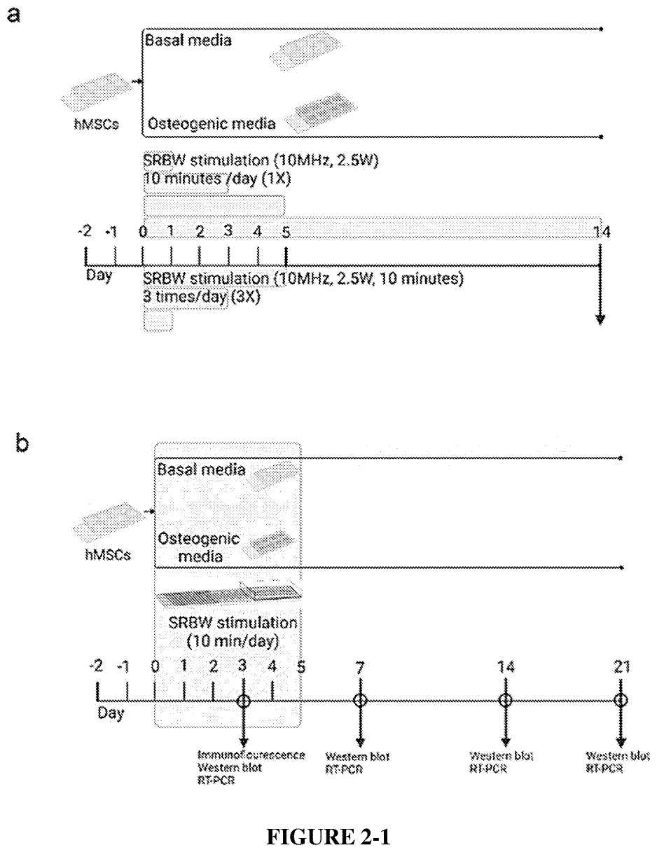

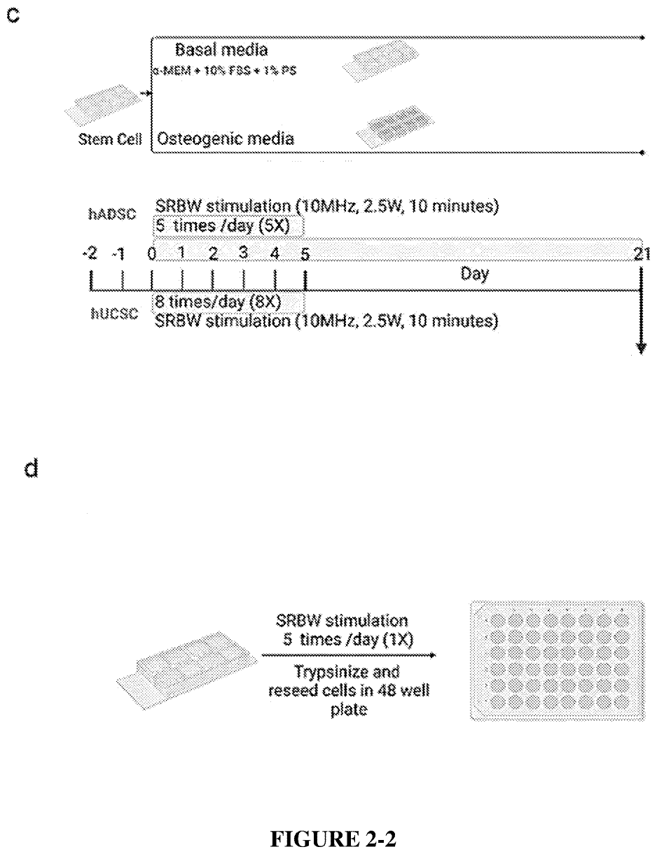

Mineralisation was assessed by alizarin red staining after 14 days of culture following the first application of the SRBW mechanostimulation (i.e., Day 16 after seeding; see workflow in FIG. 2 (a), in which 0.2% of alizarin red was dissolved in distilled water while maintaining the pH at 6.4 with ammonium hydroxide. After the stipulated incubation period, the media was aspirated and thrice washed in PBS followed by a quick rinse in distilled water. The cells were then fixed by incubating them in 100% ethanol for 15 mins followed by incubation in the alizarin red solution for 1 hr at room temperature before being imaging with light microscopy (Eclipse TS 100, Nikon Instruments Inc., Melville, NY, USA). For quantification, the stained cells were twice washed in PBS before being incubated in leaching solution (20% methanol and 10% acetic acid in distilled water) with gentle agitation for 15 mins. Absorbance was then measured at 450 nm using a spectrophotometric plate reader (CLARIOstar®, BMG LabTech, Mornington, VIC, Australia) with the clean leaching solution as a blank. The absorbance values were normalised against the protein concentration in the well.

### Immunofluorescence Staining

The cells to be fixed were washed thrice in PBS and incubated in 4% formaldehyde for 20 mins at room temperature followed by washing in PBS three times. The fixed cells were then permeabilised with 0.1% Triton™ X for 5 mins, thrice washed in PBS and then blocked by incubating them in 5% BSA in PBS for 1 hr, after which they were incubated with anti-RUNX2 (1:500), anti-COL1A1 (1:500), anti-COL2A1 (1:500) and anti-PPRA-γ (1:500) antibodies overnight at 4° C. Following washing in PBS thrice, the cells were incubated in the secondary antibody (1:1000) for 1 hr at room temperature in the dark and subsequently washed thrice in PBS. Nuclei were counterstained using Hoescht 33422 whereas actin filaments were stained with ActinRed™ 555 before imaging with confocal microscopy (A1 HD25, Nikon Instruments Inc., Melville, NY, USA). The microscope acquisition settings (laser intensity, photomultiplier gain, histogram offset, and, pinhole and image magnification) were optimised for the positive control (i.e., the untreated cells under osteogenic conditions); all images were acquired under these acquisition parameters. The low fluorescence intensity in the control images does not indicate that the protein is absent altogether in the control cells, as shown in the Western blot analysis, but simply that its expression is weak in comparison to the high expression levels in the mechanostimulated cells against which the confocal acquisition parameters were optimised to avoid overexposure. The RUNX2 nuclear-to-cytoplasmic (Nuc/Cyt) ratio, i.e., the ratio between the fluorescence intensity per unit area in the nucleus to that in the cytoplasm, was determined using ImageJ (National Institutes of Health, Bethesda, MD, USA); a ratio of 1 represents even distribution of the protein between the nucleus and cytoplasm. Cells with a Nuc/Cyt value over 1.6 are considered nuclear translocated cells (Killaars et al., 2019).

### Western Blotting

Following the stipulated incubation period (3, 5, 7, 14 or 21 days; see workflow in FIG. 2b), the SRBW treated and untreated control cells were lysed in RIPA buffer containing IX protease inhibitor, followed by incubation in reducing SDS loading buffer (62.5 mM Tris-HCl (pH 6.8), 2% SDS, 25% glycerol, 0.01% bromophenol blue and freshly added 5% β-mercaptoethanol) by heating at 95° C. for 5 mins. The denatured samples were then run on a 12% polyacrylamide gel and transferred onto a nitrocellulose membrane at 60 mV for 1 hr followed by blocking for 1 hr in 5% non-fat skimmed milk in Tris buffered saline solution (TBST; 20 mM Tris, 150 mM sodium chloride, 0.05% Tween® 20). This was then incubated overnight at 4° C. in the antibody solution (primary antibody at 1:2000 dilution and anti-rabbit antibody at 1:50,000 dilution). At the end of the incubation period, the membranes were treated with the appropriate HRP-conjugated secondary antibody in 0.05% TBST at 37° C. for 1 hr, and incubated in Pierce™ ECL Western blotting detection reagent at room temperature for 2 mins. The membranes were subsequently visualised in a gel imager (LI-COR Biotechnology, Lincoln, NE, USA); band intensities were normalised against GAPDH and the fold change was determined in comparison with the appropriate positive control.

### RNA Isolation and Real-Time Quantitative Polymerase Chain Reaction (RT-qPCR)

At the time points indicated in the workflow in FIG. 2b, total RNA content from the control and SRBW treated cells were isolated by homogenising the cells using TRiZOL™, followed by the addition of chloroform, after which the mixture was centrifuged to obtain an RNA-containing aqueous layer. The RNA was then precipitated with isopropanol and washed in ethanol, dissolved in RNAse-free water with 0.1 M EDTA and quantified using a UV spectrophotometer (NanoDrop™ One; Thermo Fisher Scientific, Waltham, MA, USA). cDNA was synthesised with the LunaScript® RT SuperMix kit and RT-qPCR carried out using the Luna® Universal qPCR Master Mix with the aforementioned primers. PP1A and β-actin were used as reference genes. As is routine in relative mRNA analysis, the relative fold change in the gene expression, which allows us to benchmark the osteogenic response of the SRBW-treated cells, is normalised against unstimulated cells in osteogenic media (positive control) that undergo optimised chemically-induced osteogenic differentiation since unstimulated cells in basal media do not express the targeted osteogenic markers and hence constitute the negative control.

### Statistics

Data presented in this study are expressed as violin plots that captured replicate measurements. Where applicable, the data was tested for normality using the Shapiro-Wilk test and analysed using a two-tailed, unpaired Student's t-test.

### Example 2: Optimisation of the SRBW Stimulation

The high frequency mechanostimulation is imparted onto the cells by coupling the SRBW irradiation through the glass-bottom of the a standard 8-well chamber culture plate slide on which the hMSCs are seeded, as shown in FIG. 1. In order to investigate the effect of the SRBW on osteogenic differentiation, cellular responses in both basal and osteogenic media conditions was explored. Unstimulated hMSCs in basal media conditions were used as the negative control whereas unstimulated hMSCS in osteogenic media were employed as the positive control in order to benchmark the osteogenic response of the mechanostimulated hMSCs.

A series of optimisation experiments was initially conducted to determine the appropriate SRBW treatment to optimise for the cell viability, morphology and calcium deposition in both basal and osteogenic conditions. Two alternative SRBW treatment regimens were applied: a daily single treatment regime (1×) comprising exposure of the hMSCs to the SRBW at 10 MHz for 10 mins at an input power of 2.5 W, or a daily triple treatment regime (3×) comprising successive 1× treatments 2 hrs apart (treatments were applied 48 hrs post seeding). 10 MHz was chosen as the excitation frequency as the SRBW does not typically exist below several MHz, and frequencies beyond 30 MHz have previously been observed to be ineffective for stimulating non-adherent cells given their shorter attenuation lengths; no significant differences in terms of osteogenic differentiation potential was observed between 10 and 30 MHz, consistent with previous work investigating various effects of the SRBW on cells (Ramesan et al., 2018; Ambattu et al., 2020; Ramesan et al., 2021). More than 90% of the SRBW stimulated cells remained viable and continued to proliferate even with 3× treatment for 5 days (FIG. 1b,c), consistent with that observed previously for high frequency acoustic excitation using SAWs (Li et al., 2009). The reason that the high frequency excitation is less detrimental to the cells, and another point of distinction of the present work in relation to preceding low frequency studies, is because cavitation—which commonly occurs in low frequency (<1 MHz) excitation and which is known to inflict considerable cell damage—is suppressed and is essentially non-existent at higher frequencies beyond several MHz (Rezk et al., 2020; Rezk et al., 2021; Yeo et al., 2014).

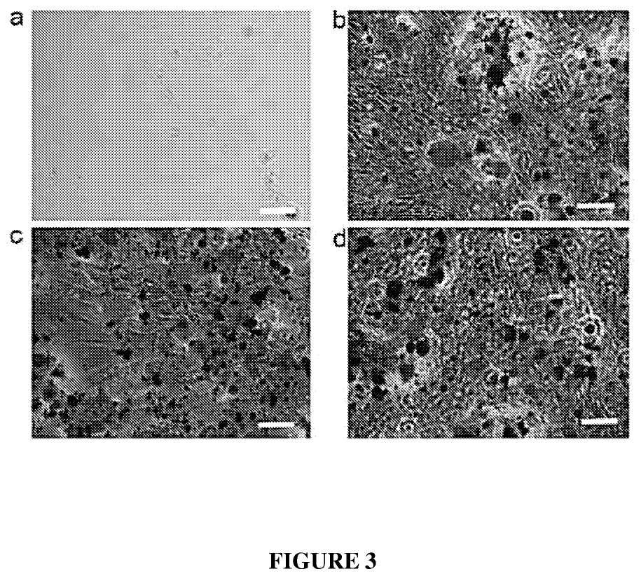

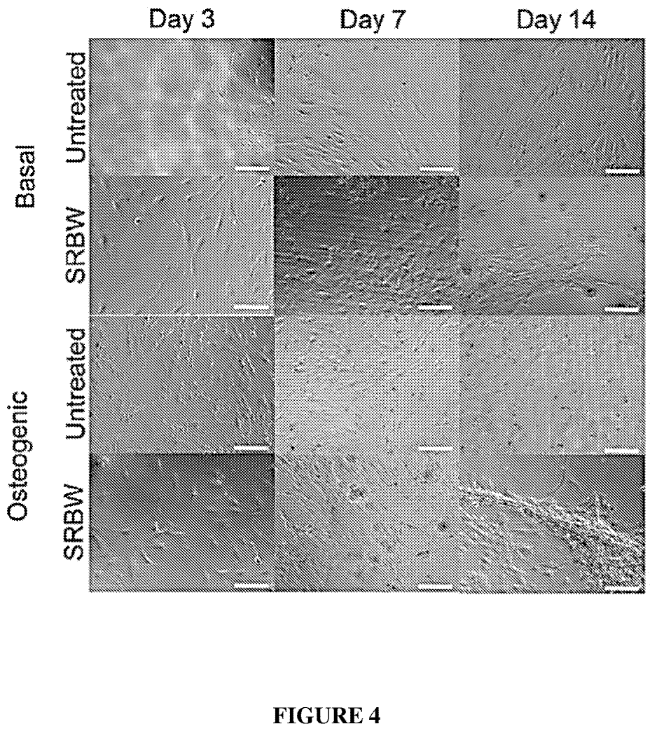

The potential for SRBW-driven osteogenesis was first assessed (see the workflow in FIG. 2a) by quantifying calcium mineralisation in the hMSCs following 14 days in culture (FIG. 2d and FIG. 3) in addition to assessing any morphological differences that arise in the cells (FIG. 4). Alizarin red staining showed a significant 6-fold increase in mineralisation for hMSCs cultured in basal conditions following 1× treatment for 5 days, and a more modest 2-fold change under osteogenic conditions. Continuing the consecutive 1× daily treatment duration from 5 days to a full 14 days did not generate further mineralisation under either basal or osteogenic conditions. Furthermore, increasing the daily treatments to 3× treatments for 5 consecutive days resulted in comparable mineralisation to the 1× treatment regime. As such, a consecutive 5-day 1× treatment regimen was deemed optimal and applied in the rest of the experiments.

### Example 3: Enhanced Osteogenic Response in SRBW-Stimulated hMSCs

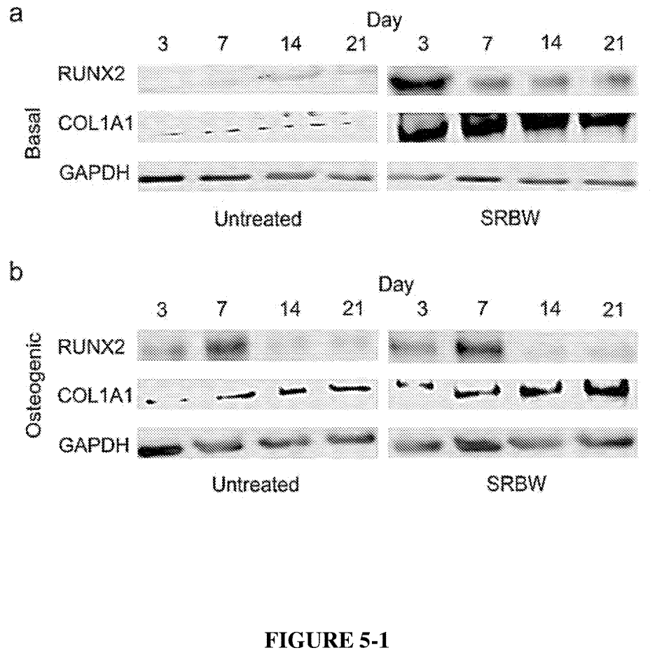

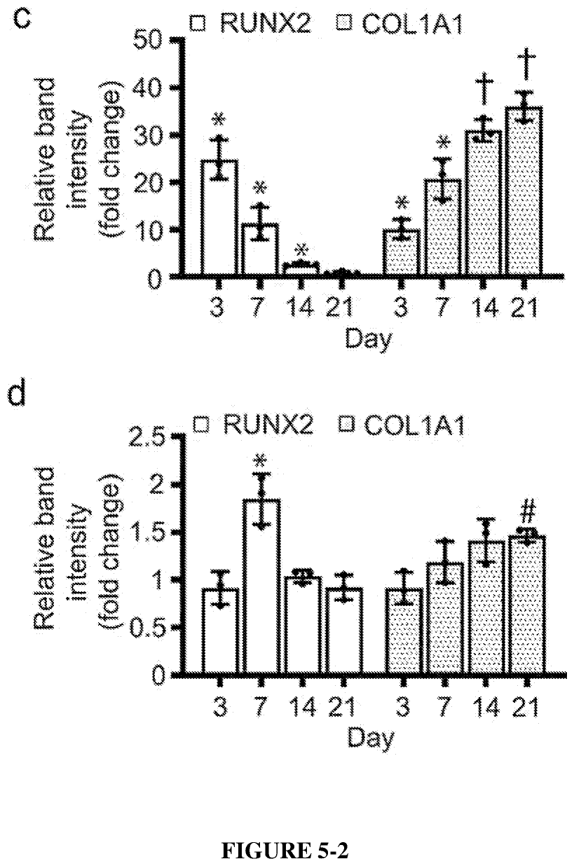

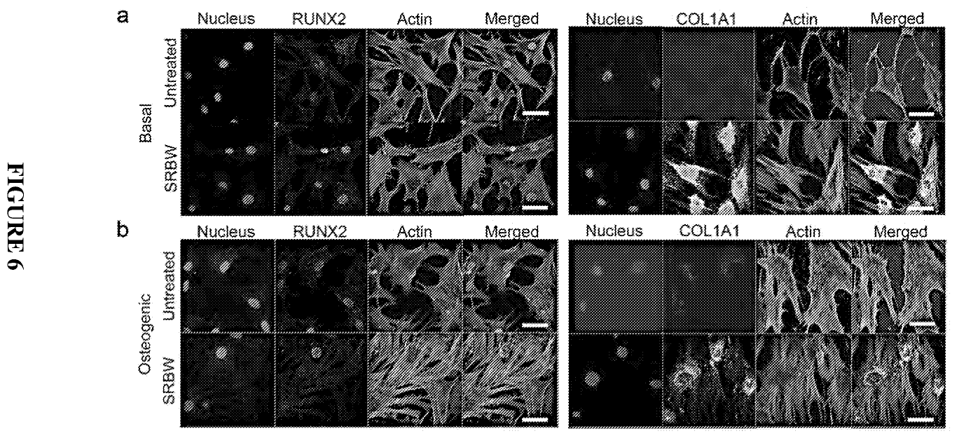

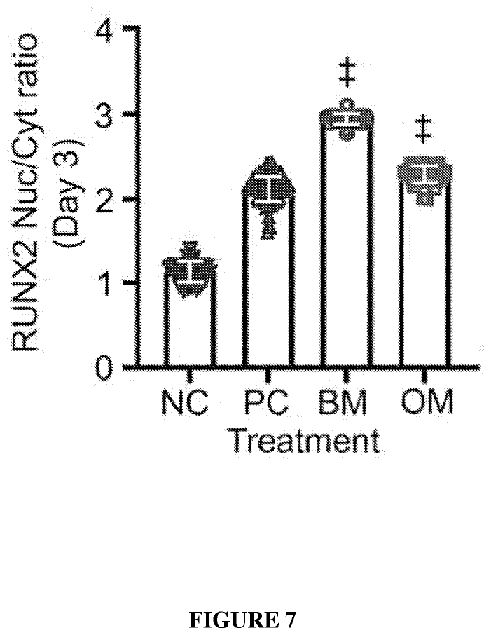

To assess the osteogenic response of the hMSCs when they are exposed to SRBW mechanostimulation, changes in the genes associated with osteogenesis were measured over a 21 day period and benchmarked against the positive control (unstimulated hMSCs under osteogenic conditions that undergo optimised chemically-induced osteogenic differentiation). The inventors observations (FIG. 5) are characteristic of typical RUNX2 (runt homology domain transcription factor 2—a marker for early osteogenic differentiation crucial for osteoblast development and bone formation (Schroeder et al., 2005; Hassan et al., 2006) that is known to be upregulated during mechanical stimulation (Kanno et al., 2007))—expression patterns in the progression through the development stages during osteogenic differentiation: upregulation within seven days following initiation of the differentiation process, followed by downregulation during late osteogenesis to the point at which expression is completely undetectable upon maturation of the osteoblast (Bagheri et al., 2018). The RUNX2 expression for the SRBW-treated hMSCs under basal conditions at Day 3 is, nevertheless, atypical (cf, for example, Galindo et al., 2005), wherein expression is generally observed to peak at Day 7 before decreasing (as observed for both treated and untreated hMSCs under osteogenic conditions). Additionally, upregulation of collagen type I alpha 1 (COL1A1), another osteogenic marker, was observed for SBRW-treated hMSCs in both basal and osteogenic conditions relative to the untreated basal hMSCs. Over the 21 day period, COL1A1 was steadily upregulated for SBRW treated hMSCs in basal conditions, in contrast to both treated and untreated hMSCs under osteogenic conditions, whose upregulation increased steadily to Day 7 before increasing at a much slower pace to 21 days. The correlation between the SBRW stimulation of the hMSCs and their early osteogenic response can also be seen from immunofluorescent imaging (FIG. 6 and FIG. 7 and FIG. 8), where clear translocation of RUNX2 from the cytoplasm to the nuclei of the SRBW-treated hMSCs under basal conditions was observed. Conspicuously, COL1A1 was weakly expressed in the control cells under both culture conditions, which is consistent with that observed in immature mesenchymal and preosteoblast cells (Aubin et al., 2002; Komori et al., 2010; Amarasekara et al., 2018; Infante et al., 2018). In contrast, COL1A1 is upregulated under both conditions upon SRBW mechanostimulation, albeit with significantly stronger expression observed in the hMSCs under basal conditions (FIG. 6). Such media-dependent difference in the RUNX2 and COL1A1 expression patterns explicitly implies the role of the SRBW mechanostimulation in inducing early markers of hMSC osteogenesis, and that the upregulation of these markers occurs over a considerably different timeframe to that observed for chemically-induced osteogenic differentiation.

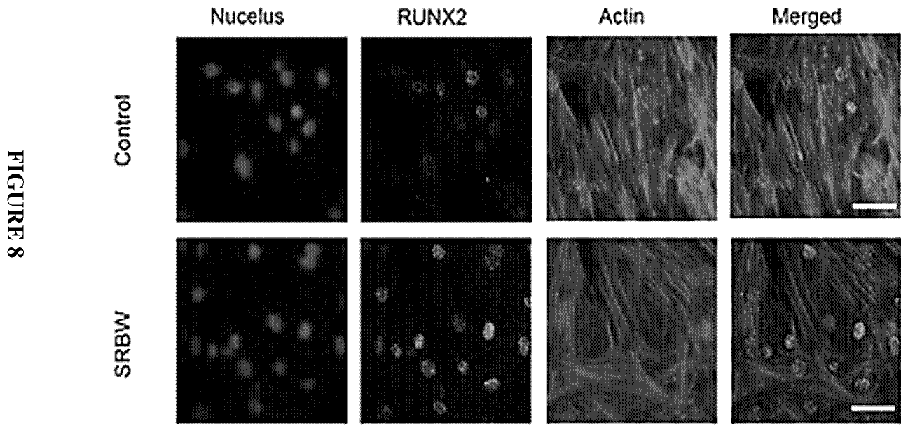

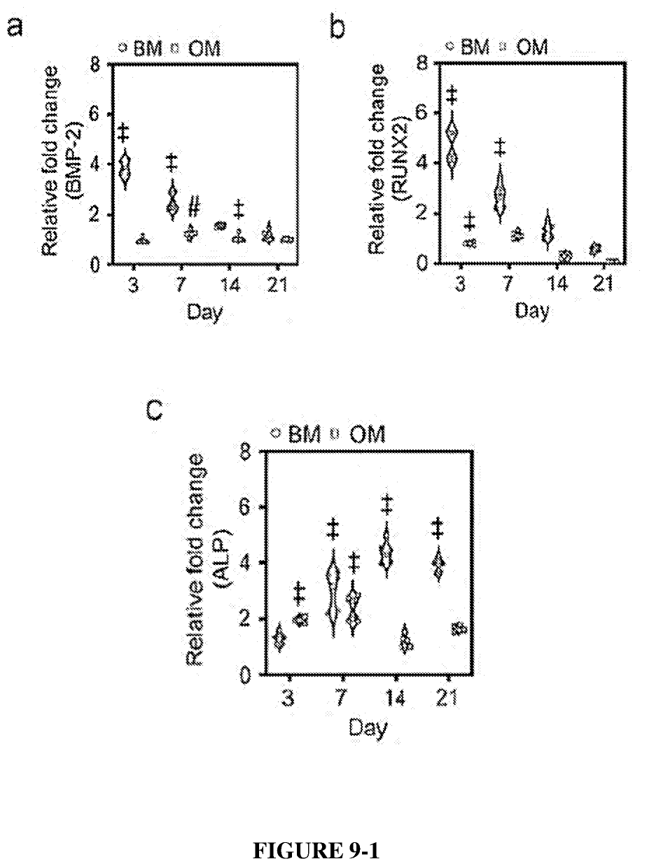

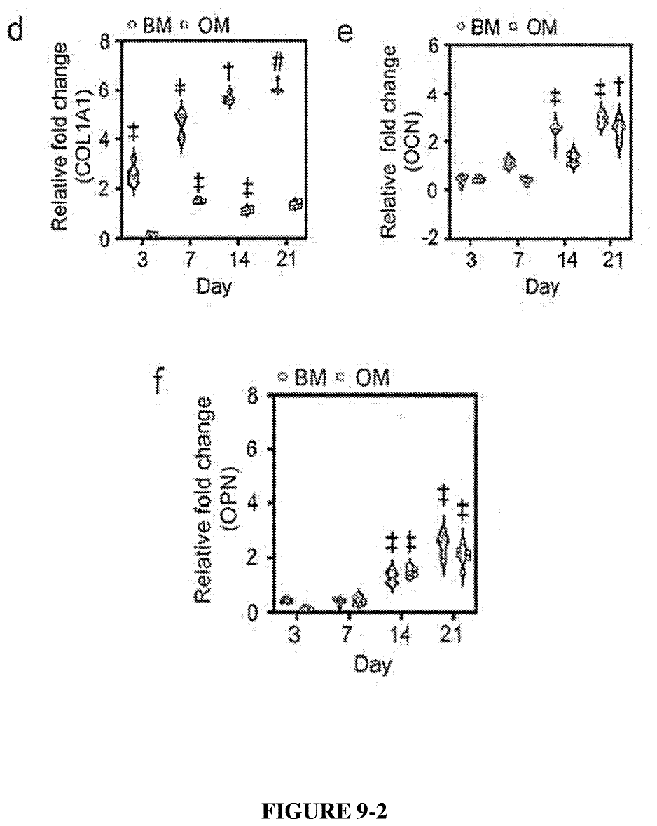

To further investigate the osteogenic potential of the SBRW mechanostimulation, a comprehensive analysis of osteogenic markers, normalised against a positive control, with real-time quantitative polymerase chain reaction (RT-qPCR) over 21 days, including bone morphogenetic protein-2 (BMP-2), alkaline phosphatase (ALP), RUNX2, COL1A1, osteocalcin (OCN) and osteopontin (OPN) was carried out. The expression of BMP-2 can be seen to increase approximately 4-fold for SRBW-treated hMSCs under basal conditions by Day 3, after which it decreased to reach the same level of expression as untreated hMSCs under the same conditions by Day 21. Under osteogenic conditions, BMP-2 expression peaked at 2-fold by Day 7, and then decreased again to the untreated expression level by Day 21 (FIG. 9). That the SRBW treatment had a significantly greater effect on BMP-2 expression in basal conditions is expected given the role of BMP-2-mediated signalling in initiating the onset of RUNX2 expression (Phimphilai et al., 2006; Jang et al., 2012; Kopf et al., 2012).

The trends for the expression of RUNX2 observed in the Western blot analysis was also consistent with the upregulation trends seen with RT-qPCR: the expression of RUNX2 for the SBRW-treated hMSCs in basal conditions being 4-fold greater than that for the untreated cells at Day 3 (FIG. 9b). The early (in addition to long-term) commitment to osteogenic lineage that is observed is quite distinct to that observed on lower frequency studies (Nikukar et al., 2013; Angle et al., 2011), all the more in light of the much shorter stimulation periods employed here (e.g., 10 mins per day as opposed to continuous excitation over days in Nikukar et al., 2013; Angle et al., 2011). Such early commitment to stem cell fate is advantageous since it avoids the transdifferentiation of stem cells from different reprogrammed sources, including many adult stem cell lines (e.g., adipose, bone marrow, dental pulp, salivary gland, synovial fluid, etc.), to other lineages (Engler et al., 2006; Zipori et al., 2004; Krabbe et al., 2005; Cherry et al., 2012; Cantone et al., 2013; Zhang et al., 2013; Bishi et al., 2013; Lee et al., 2014).

Although mechanical loading has been reported to stimulate BMP-2 expression (Sato et al., 1999), this alone does not necessarily indicate the induction of osteogenesis given that the protein also plays a role in mediating chondrogenesis (Zhou et al., 2016). Evaluation of ALP—an indicator of mineralisation during the early stages of osteoblast commitment (Prins et al., 2014)—in FIG. 9c nevertheless shows a gradual increase in relative ALP activity up to Day 14 for the SRBW treated hMSCs under basal and osteogenic conditions. COL1A1 expression, on the other hand, can be seen in FIG. 9d to be concomitantly upregulated under SRBW mechanostimulation, although more so for the cells in basal media as opposed to those in osteogenic media, in accord with the protein expression levels observed in FIG. 5.

Finally, in both media, corresponding increases in the mRNA expressions associated with other late osteogenic markers, namely, OCN (FIG. 9e), a bone-specific extracellular matrix protein secreted by osteoblasts whose expression provides a measure of its mineralisation activity, and OPN (FIG. 9f), whose enhancement in expression has been implicated in osteoblastic bone formation under mechanical stress (Morinobu et al., 2003). Such upregulation in early and late osteogenic markers in the hMSCs subjected to the SRBW mechanostimulation thus demonstrates its influence in inducing both early and long-term commitment of the stem cells to an osteogenic lineage.

### Example 4: Signal Transduction Pathway and Ion Channel Regulation Reveal Pressure and Shear Sensitive Responses

The role of the actin cytoskeleton, and, in particular, the RhoA signalling pathway, has been implicated in regulating stem cell differentiation (McBeath et al., 2004; Pemberton et al., 2015; Vogel et al., 2006). In particular, RhoA, and its downstream effector, ROCK (RhoA kinase), is known to direct commitment of mesenchymal stem cells towards osteogenic (high RhoA) or adipogenic (low RhoA) lineages. As such, the role of RhoA activation under SRBW mechanostimulation was investigated. In particular, the RT-qPCR analysis of both isoforms of these protein kinases (ROCK I and ROCK II) in FIG. 10a,b show an increase in their expressions upon SRBW mechanostimulation within a day in both culture conditions, although more mechanoresponsive under basal conditions. Expression of ROCK I, which plays a role in the formation of the actin stress fibers, can be seen to increase by 2.4-fold within a day and 4-fold by Day 3 (FIG. 10a) for the SRBW-treated hMSCs in basal media. It was only after two days following cessation of the SRBW treatment that the upregulation began to decrease at Day 7, alluding to the possibility that focal adhesion and actin stress fibre formation leading to rearrangement of the cytoskeleton, which are evident in the mechanostimulated cells in FIG. 6 and which are required to support osteoblast formation given their large polygonal morphologies (McBeath et al., 2004; Pemberton et al., 2015; Vogel et al., 2006), are triggered by the SRBW mechanostimulation. This is consistent with previous observations that report the possibility of various cellular responses, including actin stress fibre formation and cytoskeletal remodelling, being evoked by the application of acoustic pressures ranging between 0.01-0.1 MPa (Xue; et al., 2017; Stavenschi et al., 2019), the upper limit of which is comparable to the pressure typically imposed on the cell by the SRBW (~0.1 MPa).

Unlike ROCK I, the initial 4-fold increase in ROCK II expression within a day after commencement of the treatment remained steady until the mechanostimulation was relaxed, at which point the expression started to decrease to the point of downregulation by Day 7. In osteogenic media, the expression of both isoforms due to the mechanostimulation appeared to be more subdued, with expression levels ranging between 0.5- and 1.5-fold. For ROCK I, this remained upregulated without much change until Day 7 even after cessation of the stimuli at Day 5, whereas the slight increase in ROCK II expression appeared to decrease gradually after Day 1. The difference between two isoforms of ROCK proteins is that ROCK II plays a crucial role in cell adhesion, which is fundamental to cell commitment and differentiation. Favourable cellular adhesion can, in turn, induce OPN upregulation (as evident from FIG. 9).

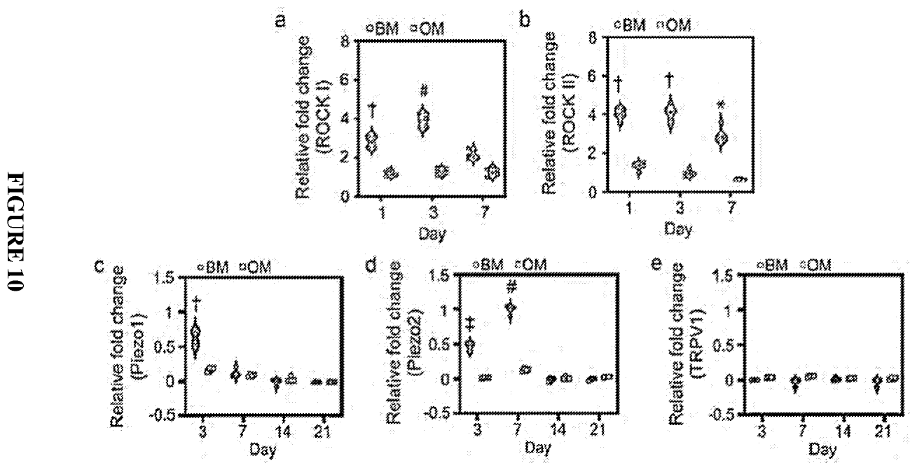

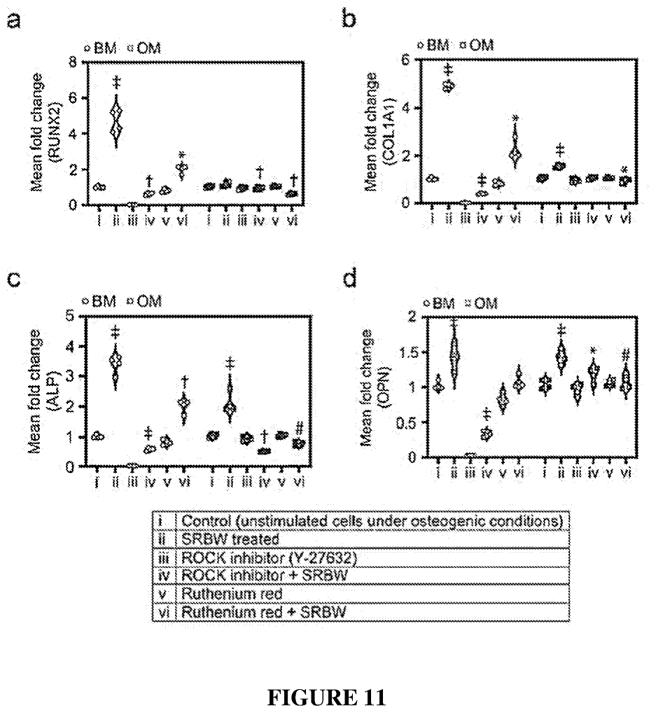

This difference in the susceptibility of various osteogenic genes to the SRBW stimulation in different culture conditions suggests that cells in basal conditions appear to be more responsive to externally-imposed mechanical cues than their counterparts under osteogenic conditions. Furthermore, addition of a ROCK inhibitor Y27632 can be seen to significantly downregulate RUNX2 and COL1A1 together with a concurrent change in ALP and OPN in the mechanostimulated cells in basal media but not in osteogenic media (FIG. 11), therefore alluding to the central role of the ROCK signalling pathway in determining the osteogenic fate in SRBW mechanostimulated hMSCs in the absence of chemical cues.

While a complete understanding of the fundamental mechanisms responsible for osteoblast differentiation remains elusive, there is mounting evidence of ROCK activation by cell-matrix adhesion complexes (Yuan et al., 2018) and the modulation of ROCK signalling by ion channel activity (Jin et al., 2019)—specifically that of mechanosensitive ion channels such as Piezo1 and Piezo2, which are believed to participate in RhoA activation in controlling the actin-myosin interactions that lead to the contraction of the actin filaments during cytoskeletal remodelling (Barzegari et al., 2020). Piezo1 has also been recognised to regulate BMP-2 expression (Sugimoto et al., 2017). As such, the influence of both Piezo1 and Piezo2 have been linked to bone formation and maintenance (Zhou et al. 2020). Additionally, knockout of Piezo1 channels in osteoblast lineage cells has been observed to disrupt osteogenesis, severely impairing bone structure and strength (Sun et al., 2019). In the same way that these excitatory cationic ion channels have been shown to be activated through various forms of mechanical forcing (Coste et al., 2010; Servin-Vences et al., 2017), the marginal increase in expression of Piezo1 and Piezo2 upon SRBW mechanostimulation of the hMSCs (FIG. 10c,d) suggests the possibility of the SRBW in regulating these ion channels; the piezo channels can however be seen to return to their ground state by the time osteogenic maturation occurred. That the SRBW might be activating these mechanosensitive ion channels, and particularly Piezo2, is corroborated by previous studies which showed SRBW mechanostimulation to incite transient membrane aberrations (Ramesan et al., 2018; Ambattu et al., 2020), especially since Piezo1 is known to be susceptible to both shear- and pressure-induced membrane stretch as well as localised membrane deformation whereas Piezo2 is activated only by membrane deformation (Taberner et al., 2019). Transient receptor potential (TRP) ion channels, specifically, TRPV1 (transient receptor potential vanilloid 1), which have more of a function as mechanosensors (Xiao et al., 2016), was found to be less sensitive to the SRBW mechanostimulation with little to no expression observed under any conditions (FIG. 10e). This distinction in responses between these various ion channels provide further evidence that the high frequency MHz-order SRBW stimulation may be generating a substantial pressure differential and shear across the hMSCs.

In the presence of a cationic ion channel blocker, ruthenium red, downregulation of RUNX2 and COL1A1 in both basal and osteogenic conditions (FIG. 11a,b) was observed, while ALP and OPN expression are seen to behave as anticipated (FIG. 11c,d). Appreciable downregulation can be seen in the hMSCs under osteogenic conditions in contrast to that seen for ROCK inhibition, where the osteogenic markers appear to maintain similar expressions as those obtained when devoid of the inhibitor (FIG. 11a,b). This suggests that the ion channels could be activating an alternative transduction pathway to ROCK in the presence of chemical stimulants; one possibility being calpain signalling. Alternatively, it is also possible that in the presence of chemical stimulants such as dexamethasone—a synthetic glucocorticoid, the SRBW, in a similar manner to that observed with other forms of mechanical stimuli (Baksh et al., 2006), could be enhancing canonical Wnt signaling, which together with the involvement of β-catenin, is known to trigger osteogenesis (Hamidouche et al., 2008).

### Example 5: Downstream Effects Triggered by Short-Term SRBW Mechanostimulation

Unlike previous studies in which the cells are often continuously stimulated for at least 7 days or more (Nikukar et al., 2013; Nikukar et al., 2016; Angle et al., 2011), the SRBW treatment that resulted in the early onset of osteogenic-determining RUNX2 upregulation under basal conditions is significantly shorter in duration (10 min per day for 5 consecutive days). While osteogenesis is a particularly intricate and complex process involving numerous signalling pathways, it is possible to postulate a possible candidate route for the osteogenic differentiation under this mechanical trigger from the protein and mRNA profiling and inhibition studies above. In contrast to the weak direct mechanical transduction imposed on the cells by low frequency bulk kHz-order mechanostimulation, this could be a result of the gating of mechanosensitive ion channels such as Piezo1 and Piezo2 as a consequence of the large pressure and shear stresses imparted on the cells by the combined in-plane and out-of-plane vibrational excitation associated with the SRBW forcing and the localised flow around the cells it generates, in a manner not dissimilar, but more intense, to that demonstrated in oscillatory shear flow setups (Arnsdorf et al., 2009; Arnsdorf et al., 2010; Govey et al., 2013. Mitogenactivated protein kinase (MAPK) and extracellular signal-regulator kinase 1/2 (ERK1/2) signalling, as downstream effectors of RhoA, are then expected to trigger a BMP-2 signalling cascade leading to upregulation of RUNX2 that promotes transcriptome alteration in the differentiation of the hMSCs into osteoblasts (Barzegari et al., 2020).

The media-dependent distinction between the osteogenic response of the hMSCs to the high frequency SRBW mechanostimulation, in particular, early upregulation in RUNX2 at Day 3 for the hMSCs under basal conditions compared with peaking of RUNX2 upregulation at Day 7, strongly suggest that chemical cues could very well be affecting the mechanotransduction relayed by the SRBW treatment. Moreover, the extensive upregulation of RUNX2 at Day 3 stimulated by the SRBW treatment could also hold important ramifications for specifically targeting osteogenic differentiation. RUNX2 is more specific than BMP-2 as an osteogenic stimulator, and hence targeting RUNX2 expression may enable a more potent approach in triggering osteogenesis of hMSCs.

### Example 6: SRBW Induces Osteogenesis in hMSCs from Different Sources

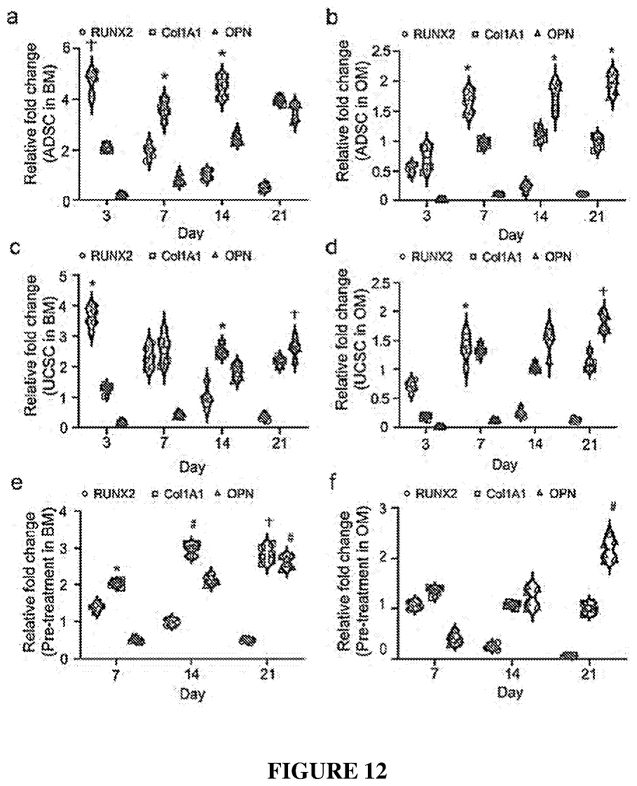

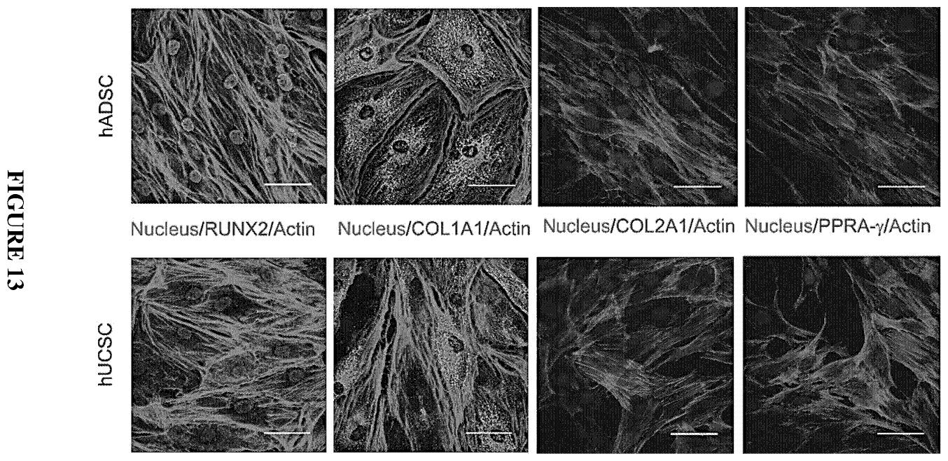

A major bottleneck in allogeneic stem cell transplantation is the limited donor material that is available. To demonstrate the versatility of the technique as a broader approach toward the design of practically translatable stem cell therapies, the osteogenic response of the stem cells beyond the bone marrow derived hMSCs examined above to other adult stem cell sources, nscandamely, hMSCs derived from adipose (hADSCs) and umbilical cord blood (hUCSC) sources, was assessed. While slightly more frequent daily stimulation stints (5× for the hADSCs and 8× for the hUCSCs; see FIG. 2c) over the same 5-day period, these still represent relatively short treatment durations compared to the continuous excitation over longer periods (>7 days) that have been reported for low frequency mechanostimulation treatments (Nikukar et al., 2013; Angle et al., 2011). Moreover, the early onset of RUNX2 expression previously reported for bone marrow derived hMSCs upon SRBW mechanostimulation was also observed for hADSCs and hUCSCs under basal conditions, resulting in increased expression of COL1A1 and OPN (FIG. 12a,c). Under osteogenic conditions, enhanced expression of these osteogenic markers in both tissue tissue sources was observed similar to that found for the bone marrow derived hMSCs (FIG. 12b,d). Notably, the SRBW, while triggering osteogenic differentiation, did not initiate adipogenesis, as determined from the lack of peroxisome proliferator-activated receptor gamma (PPRA-γ) expression, or chondrogenesis, inferred from the absence of collagen type II alpha 1 (COL2A1), in these cells under basal conditions (FIG. 13).

Additionally, the inventors checked to ensure that a SRBW pre-treatment regimen involving the short 1× treatment over 5 days prior to reseeding of the hMSCS onto a 48-well plate (FIG. 2d) retains their early lineage commitment, as supported by the increased expression in the late osteogenic marker OPN (FIG. 12e,f). Such preservation of the osteogenic differentiation potential in the reseeded hMSCs therefore presents an exciting new pathway for the pre-treatment of stem cells and practical large-scale regeneration for bone tissue engineering.

### REFERENCES

Amarasekara et al. (2018) Immune Network 18:e8.
Ambattu et al. (2020) Communications Biology 3: 1-9.
Angle et al. (2011) Ultrasonics 51: 281-288.
Arnsdorf et al. (2010) Journal of Biomechanics 43: 2881-2886.
Arnsdorf et al. (2009), Journal of Cell Science 122: 546-553.
Aubin et al. (2002) (Eds.), Principles of Bone Biology, 2nd Edition, Elsevier, London, 59-81.
Bagheri et al. (2018) Journal of Tissue Engineering and Regenerative Medicine 12 (2) 304-315.
Baksh et al. (2006) Transactions of the 52nd Annual Meeting of the Orthopaedic
Research Society, 0131.
Barzegari et al. (2020) Cell and Tissue Research 381: 1-12.
Bishi et al. (2013) Journal of Materials Chemistry B 1: 3972-3984.
Cantone et al. (2013) Nature Structural and Molecular Biology 20: 282-289.
Cherry et al. (2012) Cell 148: 1110-1122.
Coste et al. (2010) Science 330: 55-60.
Engler et al. (2006) Cell 126: 677-689.
Galindo et al. (2005) Journal of Biological Chemistry 280: 20274-20285.
Garman et al. (2007) Journal of Orthopaedic Research 25: 732-740.
Govey et al. (2013) Current Osteoporosis Reports 11: 83-91.
Hamidouche et al. (2008) The FASEB Journal 22: 3813-3822.
Hassan et al. (2006) Journal of Biological Chemistry 281: 40515-40526.
Holick (1998) Bone 22: 105S-111S.
Infante et al. (2018) Stem Cell Research and Therapy 9: 244.
Jang et al. (2012) Journal of Biological Chemistry 287: 905-915.
Jin et al. (2019) American Journal of Physiology-Heart and Circulatory Physiology 296: H908-H909.
Kanno et al. (2007) Journal of Cellular Biochemistry 101: 1266-1277.
Killaars et al. (2019) Advanced Science 6: 1801483.
Komori et al. (2010) Cell and Tissue Research 339: 189-195.
Kopf et al. (2012) BMC Biology 10: 37.
Krabbe et al. (2005) APMIS: Acta Pathologica, Microbiologica, et Immunologica Scandinavica 113: 831-844.
Lee et al. (2014) Scientific Reports 4: 1-8.
Li et al. (2009) Biomicrofluidics 3: 034102.
McBeath et al. (2004) Developmental Cell 6: 483-495.
Morinobu et al. (2003) Journal of Bone and Mineral Research 18: 1706-1715.
Nikukar et al. (2016) Journal of Biomedical Nanotechnology 12: 1478-1488.
Nikukar et al. (2013) ACS Nano 7: 2758-2767.
Pemberton et al. (2015) Nanomedicine 10: 547-560.
Phimphilai et al. (2006) Journal of Bone and Mineral Research 21: 637-646.
Prins et al. (2014) Stem Cell Research 12: 428-440.
Ramesan et al. (2018) Nanoscale 10: 13165-13178.
Ramesan et al. (2021) ACS Applied Bio Materials 4: 2781-2789.
Rezk et al. (2021) Advanced Science 8: 2001983.
Rezk et al. (2020) The Journal of Physical Chemistry Letters 11: 4655-4661.
Sato et al. (1999) Journal of Bone and Mineral Research 14: 1084-1095.
Schroeder et al. (2005) Birth Defects Research Part C: Embryo Today: Reviews 75: 213-225.
Servin-Vences et al. (2017) eLife 6 e21074.
Stavenschi et al. (2019) The FASEB Journal 33: 4178-4187.
Sugimoto et al. (2017) Scientific Reports 7: 1-14.
Sun et al. (2019) eLife 8 e47454.
Taberner et al. (2019) Proceedings of the National Academy of Sciences USA 116: 14260-14269.
Thellin et al. (1999) Journal of Biotechnology 75: 291-295.
Vogel et al. (2006) Nature Reviews Molecular Cell Biology 7: 265-275.
Xiao et al. (2016) Stem Cell Research and Therapy 7: 140.
Xue et al. (2017) Biomaterials 134: 22-30.
Yeo et al. (2014) Annual Review of Fluid Mechanics 46: 379-406.
Yuan et al. (2018) Investigative Ophthalmology & Visual Science 59: 3619-3629.
Zhang et al. (2013) Biomaterials 34: 3962-3969.
Zhang et al. (2017) Small 13: 1602962.
Zhou et al. (2016) Cell and Tissue Research 366: 101-111.
Zhou et al. (2020) eLife 9: e52779.
Zipori (2004) Molecules and Diseases 33: 211-215.

1. A method of differentiating a population of mesenchymal stem cells towards an osteogenic lineage, the method comprising exposing the population of stem cells to intermittent short duration acoustic wave energy, wherein the acoustic wave energy is provided at a frequency greater than 3 MHz.

2. The method of claim 1, wherein the acoustic wave energy is provided at a frequency between greater than 3 MHz and about 35 MHz.

3. The method of claim 1 or 2, wherein the acoustic wave energy is provided at a frequency 5 MHz and about 15 MHz or 25 MHz to 35 MHz.

4. The method of any one of claims 1 to 3, wherein the acoustic wave energy is provided at a frequency of about 10 MHz.

5. The method of any one of claims 1 to 4, wherein the acoustic wave energy is provided in the form of surface reflected bulk waves (SRBW).

6. The method of any one of claims 1 to 4, wherein the acoustic wave energy is provided in the form of surface acoustic waves (SAW).

7. The method of any one of claims 1 to 6, wherein the mesenchymal stem cells are bone marrow derived mesenchymal stem cells, adipose derived mesenchymal stem cells, or umbilical cord derived mesenchymal stem cells.

8. The method of any one of claims 1 to 7, wherein the intermittent exposure comprises exposing the population of cells to a one or more periods of acoustic wave energy followed by incubation in the absence of acoustic stimulation for one or more consecutive days.

9. The method of any one of claims 1 to 8, wherein the short duration comprises a period of between about 3 minutes and about 20 minutes.

10. The method of claim 9, wherein the cells are exposed to acoustic wave energy for a period of about 10 minutes.

11. The method of any one of claims 1 to 10, wherein the cells are exposed to acoustic wave energy for a short duration period once, twice, 3 times, 4 times, 5 times, 6 times, 7 times, 8 times, 9 times, or 10 times per day.

12. The method of claim 11, wherein the cells are exposed to acoustic wave energy for a short duration period once per day.

13. The method of any one of claims 1 to 12, wherein the cells are exposed to acoustic wave energy for a short duration period for at least 3 days, for at least 4 days, or for at least 5 days.

14. The method of claim 13, wherein the cells are exposed to acoustic wave energy for at least 5 days.

15. The method of any one of claims 1 to 14, wherein the cells express at least one osteogenic marker after at least 3 consecutive days of exposure to acoustic wave energy.

16. The method of claim 15, wherein the osteogenic marker is selected from group comprising RUNX2, COL1A1, and OCN.

17. The method of any one of claims 1 to 16, wherein the cells are harvested on the fifth consecutive day of exposure to acoustic wave energy.

18. The method of any one of claims 1 to 17, wherein the acoustic wave energy is provided to the population of cells via an apparatus, the apparatus comprising:
(i) an acoustic wave generator configured to generate acoustic energy at a selected power and frequency; and
(ii) a receptacle for accommodating a population of cells in a culture medium, the receptacle being configured to receive acoustic energy generated by the acoustic wave generator.

19. The method of claim 18, wherein at least part of the acoustic wave generator is arranged in direct contact with the culture medium.

20. The method of claim 18, wherein the acoustic wave generator is separated from the culture medium.

21. The method of any one of claims 18 to 20, wherein the receptacle defines a reservoir configured to accommodate the population of cells in a culture medium.

22. The method of any one of claims 18 to 21, wherein the acoustic wave generator comprises a piezoelectric substrate defining a working surface and an interdigitated transducer located on and in contact with the working surface of the piezoelectric substrate.

23. The method of any one of claims 18 to 22, wherein the receptacle is coupled to the acoustic wave generator with a coupling material.

24. The method of claim 22 or 23, wherein the acoustic wave energy is propagated as a surface acoustic wave (SAW) along the working surface.

25. The method of claim 22 or 23, wherein the acoustic wave energy is propagated as a surface reflected bulk wave (SRBW) within the piezoelectric substrate and internally reflected between the working surface and an adjacent surface of the piezoelectric substrate.

26. The method of any one of claims 1 to 25, wherein the frequency of the applied acoustic energy is in the range of about 5 MHz to about 35 MHz.

27. The method of any one of claims 1 to 26, wherein the frequency of the applied acoustic energy is in the range of about 5 MHz to about 15 MHz or 25 MHz to 35 MHz.

28. The method of claim 27, wherein the frequency of the applied acoustic energy is about 10 MHz.

29. The method of any one of claims 18 to 28, wherein the input power for the acoustic wave generator is in the range of about 1 W to about 10 W.

30. The method of claim 29, wherein the input power for the acoustic wave generator is about 2.5 W.

31. The method of any one of claims 1 to 30, wherein an acoustic pressure applied to the cells by the acoustic wave generator is between about 0.01 MPa and 1 MPa, preferably about 0.1 MPa.

32. The method of any one of claims 18 to 31, wherein the cell culture media comprises a basal media.

33. The method of any one of claims 18 to 31, wherein the cell culture media comprises an osteogenic media.

34. A population of osteogenic-committed stem cells when produced by the method of any one of claims 1 to 33.

35. The population of osteogenic-committed stem cells of claim 34, wherein the osteogenic-committed cells are osteoblasts.

36. The population of osteogenic-committed stem cells of claim 34 or 35 for use in the treatment of a bone disease or disorder.

37. A method of treating a bone disease or disorder, the method comprising administering a population of osteogenic-committed stem cells produced by the method of any one of claims 1 to 33.

38. The method of claim 37, wherein the population of osteogenic-committed stem cells are obtained from autologous stem cells.

39. The method of claim 37, wherein the population of osteogenic-committed stem cells are obtained from allogenic stem cells.

40. The method of any one of claims 37 to 39, wherein the bone disease or disorder is selected from the group comprising osteoporosis, osteoarthritis, bone fracture, osteomyelitis, and osteonecrosis.
